(19) United States
(12) Patent Application Publication
(10) Pub. No.: US 2021/0169856 A1
(43) Pub. Date: Jun. 10, 2021

(54) FORMULATIONS COMPRISING DOPAMINE-B-HYDROXYLASE INHIBITORS AND METHODS FOR THEIR PREPARATION

(71) Applicant: BIAL - PORTELA & CA, S.A., S Mamede de Coronado (PT)

(72) Inventors: Pedro Miguel DA COSTA BARROCAS, S. Mamede do Coronado (PT); Sandra DA SILVA TEIXEIRA BASTOS, S. Mamede do Coronado (PT); Teófilo CARDOSO DE VASCONCELOS, S. Mamede do Coronado (PT); Ricardo Jorge DOS SANTOS LIMA, S. Mamede do Coronado (PT); Ana Rita GONÇALVES DA CRUZ RAMOS PIRES, S. Mamede do Coronado (PT); Raquel QUEIROZ PORTELA MENDES FIGUEIREDO, S. Mamede do Coronado (PT); Jaison CARLOSSO MACHADO, S. Mamede de Coronado (PT)

(21) Appl. No.: 17/254,962

(22) PCT Filed: Jun. 24, 2019
(86) PCT No.: PCT/PT2019/050018

§ 371 Date: Dec. 22, 2020

(30) Foreign Application Priority Data
Jun. 25, 2018 (GB) 1810395.2

(51) Int. Cl. A61K 31/4178; A61K 9/20; A61K 9/48; A61K 9/00

(52) U.S. Cl. CPC A61K 31/4178; A61K 9/2018; A61K 9/2054; A61K 9/0053; A61K 9/4858; A61K 9/4866; A61K 9/485; A61K 9/2009

(57) ABSTRACT

The present invention relates to formulations comprising Compound X or a pharmaceutically acceptable salt or solvate thereof:

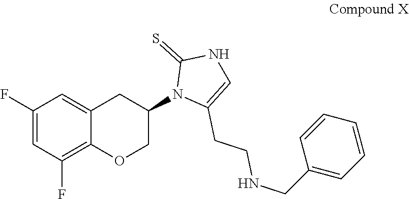 [SMILES: Fc1cc(F)c2c(c1)C[C@@H](n1c(CCNCc3ccccc3)c[nH]c1=S)CO2]

and methods for their preparation.

### CROSS REFERENCE TO RELATED APPLICATIONS

This application is a filing under 35 U.S.C. 371 of International Application No. PCT/PT2019/050018, filed Jun. 24, 2019, entitled “FORMULATIONS COMPRISING DOPAMINE-β-HYDROXYLASE INHIBITORS AND METHODS FOR THEIR PREPARATION,” which claims priority to Great Britain Patent Application No. 1810395.2, filed Jun. 25, 2018, which applications are incorporated by reference herein in their entirety.

### FIELD OF THE INVENTION

This invention relates to pharmaceutical formulations and methods for their preparation. In particular, this invention relates to formulations comprising inhibitors of dopamine-β-hydroxylase and methods for their preparation. More particularly, this invention relates to formulations comprising Compound X or a pharmaceutically acceptable salt or solvate thereof:

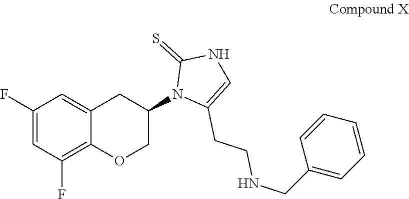 [SMILES: Fc1cc(F)c2c(c1)C[C@@H](n1c(CCNCc3ccccc3)c[nH]c1=S)CO2]

and methods for their preparation.

### BACKGROUND OF THE INVENTION

Compound X is a dopamine-β-hydroxylase inhibitor. Potent dopamine-β-hydroxylase inhibitors having high potency and significantly reduced brain access are disclosed in WO 2008/136695. WO 2008/136695 describes compounds of formula I:

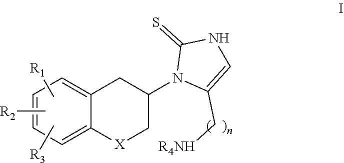

where R₁, R₂ and R₃ are the same or different and signify hydrogens, halogens, alkyl, nitro, amino, alkylcarbonylamino, alkylamino or dialkylamino group; R₄ signifies -alkylaryl or -alkylheteroaryl; X signifies CH₂, oxygen atom or sulphur atom; n is 2 or 3; including the individual (R)- and (S)-enantiomers or mixtures of enantiomers thereof; and including pharmaceutically acceptable salts and esters thereof, wherein the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyl, alkyloxy, halogen or nitro group; the term halogen means fluorine, chlorine, bromine or iodine; the term heteroaryl means heteroaromatic group. In particular, WO 2008/136695 discloses Compound X:

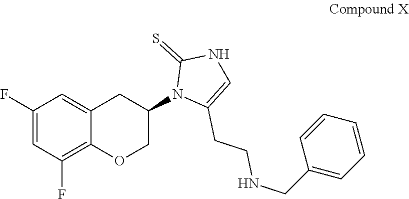 [SMILES: Fc1cc(F)c2c(c1)C[C@@H](n1c(CCNCc3ccccc3)c[nH]c1=S)CO2]

Processes for the preparation of compounds of formula I, and in particular Compound X, are described in WO 2008/136695, WO 2013/002660 and WO 2015/038022 and are incorporated by reference herein.

WO 2014/077715 discloses Compound X for use in treating pulmonary arterial hypertension, either when administered alone or in combination with a further active pharmaceutical ingredient, for example bosentan.

Formulations comprising Compound X generally have poor flowability, which leads to difficulties in preparing tablets. It is also desirable that tablets are uniform in colour, for example without spots of the active ingredient being visible. This is important for blinding in clinical studies, as well as for good patient compliance with treatment and aesthetic/commercial reasons. With formulations of homogenous colour it may also be easier to detect instability problems since changes in the homogenous colour could highlight chemical degradation/instability of the excipients and/or drug. Further, Compound X is insoluble in water, which leads to difficulties in preparing a formulation comprising this compound. The inventors have developed formulations and methods of preparing formulations of Compound X with improved dissolution.

### SUMMARY OF THE INVENTION

The present invention relates to formulations comprising inhibitors of dopamine-(3-hydroxylase having high potency and significantly reduced brain access, and methods for the preparation of such formulations. In particular, the present invention relates to pharmaceutical formulations comprising Compound X or a pharmaceutically acceptable salt or solvate thereof:

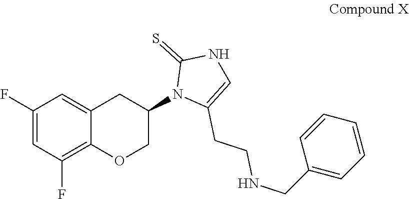 [SMILES: Fc1cc(F)c2c(c1)C[C@@H](n1c(CCNCc3ccccc3)c[nH]c1=S)CO2]

and methods of preparation of formulations comprising Compound X or a pharmaceutically acceptable salt or solvate thereof.

Compound X is insoluble in water.

In this specification, we are adopting the definitions of solubility from the classification in the European Pharmacopeia 6^{th} Edition and the United States Pharmacopeia 33 (Reference Tables: Description and Solubility):

| | Parts of Solvent Required |
|---|---|
| Descriptive Term | for 1 Part of Solute |
| Very soluble | Less than 1 |
| Freely soluble | From 1 to 10 |
| Soluble | From 10 to 30 |
| Sparingly soluble | From 30 to 100 |
| Slightly soluble | From 100 to 1000 |
| Very slightly soluble | From 1000 to 10,000 |
| Practically insoluble, or | 10,000 and over |
| Insoluble | |

The inventors have found that in aqueous medium the solubility of compound X decreases as the pH increases; and maximum solubility is achieved by using HCl 0.01 (pH 2.0). Further increase in pH results in solubility decrease. Compound X is also insoluble in isopropanol, iso-octane and cyclohexane and practically insoluble in ethanol. It is very slightly soluble in ethyl acetate, acetonitrile, chloroform, isopropyl acetate, toluene and methanol. It is slightly soluble in acetone, dichloromethane and methyl ethyl ketone; and sparingly soluble in dimethylformamide, dimethyl sulfoxide, N,N-methylpyrrolidone, tetrahydrofuran, acidified acetonitrile, acidified methanol, as well as acidified water (pH 1.2-2.0). The pH-dependent solubility profile of Compound X presents certain challenges to the development of pharmaceutical formulations, namely for its dissolution.

### BRIEF DESCRIPTION OF THE DRAWINGS

FIGS. 1 to 4 illustrate the results of an investigation into the effect of compression force and punch shape on formulation characteristics of tablets comprising Compound X.

FIG. 1: shows Oblong tablet vs Round tablet compression parameters evaluation (thickness and friability) at a machine speed of 10 RPM. The key is as follows:

[symbol] Thickness

Round Tablets

(mm)

[symbol] Friability

Round Tablets

(%)

[symbol] Thickness

Oblong

Tablets (mm)

[symbol] Friability

Oblong

Tablets (%)

FIG. 2 shows Oblong tablet vs Round tablet compression parameters evaluation (average weight, hardness and disintegration time) at a machine speed of 10 RPM. The key is as follows:

[symbol] Average

weight Round

Tablets (mg)

[symbol] Hardness

Round Tablets

(N)

[symbol] Disintegration

time Round

Tablets (s)

[symbol] Average

weight Oblong

Tablets (mg)

[symbol] Hardness

Oblong

Tablets (N)

[symbol] Disintegration

time Oblong

Tablets (s)

FIG. 3 shows the speed challenge oblong tablet compression parameters evaluation (thickness and friability) at a compression force of 17 KN. The key is as follows:

[symbol] Thickness

(mm)

[symbol] Friability (%)

FIG. 4 shows the speed challenge oblong tablet compression parameters evaluation (average weight, hardness and disintegration time) at a compression force of 17 KN. The key is as follows:

[symbol] Average

weight (mg)

[symbol] Hardness (N)

[symbol] Disintegration

time (s)

### DETAILED DESCRIPTION OF THE INVENTION

The present invention provides a pharmaceutical formulation comprising Compound X or a pharmaceutically acceptable salt or solvate thereof in combination with one or more pharmaceutically acceptable excipients.

Compound X may be present in the formulations of the present invention in free base form or in the form of a pharmaceutically acceptable salt. Suitable pharmaceutically acceptable salts of Compound X include acid addition salts, for example, Compound X in the form of a hydrochloride salt. Other suitable acid addition salts include, but are not limited to, L-tartrate, mesylate, tosylate, trifluoroacetate, citrate, glycolate, oxalate and acetate salts. Suitable solvated forms of Compound X include hydrated forms.

Suitable pharmaceutically acceptable excipients include, but are not limited to, one or more fillers, lubricants, disintegrants, binders, colouring agents and any combination thereof.

In one aspect, the present invention provides a pharmaceutical formulation comprising Compound X or a pharmaceutically acceptable salt or solvate thereof in combination with one or more pharmaceutically acceptable excipients, wherein the formulation exhibits a dissolution of at least about 50% at about 45 minutes, preferably at a temperature of about 37° C.±0.5° C. and a pH of about 4.5+0.5% sodium lauryl sulphate, using a paddle apparatus, preferably at a speed of about 100 rpm. More preferably, the formulation exhibits a dissolution of at least about 60% at about 45 minutes at a temperature of about 37° C.±0.5° C. and a pH of about 4.5+0.5% sodium lauryl sulphate using a paddle apparatus at a speed of about 100 rpm. Most preferably, the formulation exhibits a dissolution of at least about 70% at about 45 minutes at a temperature of about 37° C.±0.5° C. and a pH of about 4.5+0.5% sodium lauryl sulphate using a paddle apparatus at a speed of about 100 rpm.

Typically, the formulation comprises Compound X or a pharmaceutically acceptable salt or solvate thereof in combination with at least one filler and at least one further excipient. The filler may be selected from the following group: microcrystalline cellulose (such as MCC 101, Avicel® PH 101 or Avicel® PH 102), anhydrous lactose, co-processed 75% microcrystalline cellulose and 25% lactose (for example Cellactose® 80 or Microcelac 100), isomalt (for example GaleniQ® 801), Emcompress® (dibasic dihydrate calcium phosphate), ammonium alginate, calcium carbonate, calcium lactate, dibasic anhydrous calcium phosphate, tribasic calcium phosphate, calcium silicate, calcium sulfate, carbomer, carboxymethylcellulose calcium, carboxymethylcellulose sodium, cellulose, silicified microcrystalline cellulose, cellulose acetate, compressible sugar, ceratonia, chitosan, corn starch, pregelatinized starch (for example Starch 1500), dextrates, dextrin, dextrose, erythritol, ethylcellulose, fructose, fumaric acid, glyceryl monooleate, glyceryl monostearate, glyceryl palmitostearate, hydroxyethyl cellulose, hydroxyethylmethyl cellulose, hydroxypropyl betadex, hydroxypropyl cellulose, hydroxypropyl starch, hypromellose, hypromellose acetate succinate, kaolin, lactitol, lactose, lactose monohydrate, lactose, monohydrate and corn starch (for example StarLac), lactose, monohydrate and povidone (for example Ludipress), magnesium carbonate, magnesium oxide, maltitol, maltodextrin, maltose, mannitol, medium chain triglycerides, methylcellulose, pectin, polaxamer, polycarbophil, polydextrose, poly (DL-lactic acid), polyethylene glycol, polyethylene oxide, polymethacrylates, polyoxyglycerides, polyvinyl alcohol, shellac, simethicone, sodium alginate, sodium chloride, sorbitol, starch, sucrose, sugar spheres, sulfobutylether B-cyclodextrin, talc, tragacanth, titanium dioxide, trehalose, microcrystalline wax, white wax, yellow wax, xantham gum, xylitol, zein and combinations thereof. Preferably, the filler is lactose, microcrystalline cellulose, dibasic dihydrate calcium phosphate, isomalt, mannitol or any combination thereof. The filler is suitably present in an amount from about 1 to about 97 wt % of the total weight of the formulation.

The formulation may comprise Compound X or a pharmaceutically acceptable salt or solvate thereof in combination with at least one lubricant and optionally at least one further excipient. The or each lubricant may be selected from the group consisting of: calcium stearate, colloidal silicon dioxide, glyceryl behenate, glyceryl monostearate, glyceryl palmitostearate, leucine, magnesium oxide, magnesium silicate, magnesium stearate, magnesium lauryl sulfate, magnesium trisilicate, medium-chain triglycerides, mineral oil, myristic acid, palmitic acid, polaxamer, polyethylene glycol, potassium benzoate, sodium benzoate, sodium lauryl sulfate, sodium stearyl fumarate, stearic acid, talc, hydrogenated vegetable oil, hydrogenated castor oil, light mineral oil and zinc stearate, and combinations thereof. Preferably, the lubricant is a glidant. Preferably, the lubricant is magnesium stearate. The lubricant is suitably present in an amount from about 0.1 to about 10 wt % of the total weight of the formulation.

Suitably, the pharmaceutical formulation comprises a mixture of two or more fillers. For example, the filler may be a mixture of anhydrous lactose and microcrystalline cellulose. The filler may be a mixture of lactose (for example Lactose 200M) and microcrystalline cellulose (for example MCC 101). The filler may be a mixture of microcrystalline cellulose (for example MCC 101) and dibasic dihydrate calcium phosphate (for example Emcompress®). The filler may be a mixture of microcrystalline cellulose (for example MCC 101) and isomalt (for example GaleniQ® 801).

Suitably, the pharmaceutical formulation comprises a mixture of two or more fillers, wherein at least one filler is a plastic deformable filler and at least one filler is a brittle filler. Examples of plastic deformable fillers are microcrystalline cellulose (for example MCC 101, Avicel® PH 101, Avicel® PH 102), starch, cellulose acetate, and maltodextrin. Examples of brittle fillers are mannitol, anhydrous lactose, lactose, dibasic dihydrate calcium phosphate (for example Emcompress®), starch, pregelatinized starch (for example Starch 1500), and isomalt. Suitably, the pharmaceutical formulation comprises a mixture of two fillers, wherein one filler is a plastic deformable filler and the other filler is a brittle filler. Suitably, the filler is a mixture of microcrystalline cellulose (for example MCC 101) and mannitol. Alternatively, the filler may be a mixture of microcrystalline cellulose (for example MCC 101) and isomalt. Alternatively, the filler may be a mixture of microcrystalline cellulose (for example MCC 101) and pregelatinized starch (for example Starch 1500). Preferably, the filler is a mixture of microcrystalline cellulose (for example MCC 101) and isomalt.

One possible method for characterising a plastic deformable filler and a brittle filler is to place both types of fillers, separately, under compression in a tableting machine. A constant compression form should be maintained, whilst increasing the compression speed. The plastic deformable filler will result in a decrease in the tablet hardness. However, the brittle filler will result in the hardness of the tablets staying constant or slightly decreasing, when exposed to an increase of the compression speed. Other known methods may also be employed.

The formulation may comprise Compound X or a pharmaceutically acceptable salt or solvate thereof in combination with at least one disintegrant and optionally at least one further excipient. The disintegrant may be alginic acid, calcium alginate, carboxymethylcellulose calcium, carboxymethylcellulose sodium, cellulose, chitosan, colloidal silicon dioxide, corn starch, pregelatinized starch, docusate sodium, glycine, guar gum, hydroxypropyl cellulose, magnesium aluminum silicate, methylcellulose, microcrystalline cellulose, polacrilin potassium, povidone, sodium alginate, crospovidone, sodium croscarmellose or sodium starch glycolate, or mixtures thereof. Preferably, the disintegrant is crospovidone. The disintegrant is suitably present in an amount from about 0.1 to about 30 wt % of the total weight of the formulation. More preferably the disintegrant is present in an amount from about 1 to about 20 wt % and more preferably in an amount from about 2 to about 15 wt % of the total weight of the formulation.

The formulation may comprise Compound X or a pharmaceutically acceptable salt or solvate thereof in combination at least one binder and optionally at least one further excipient. The binder may be acacia, agar, alginic acid, calcium carbonate, calcium lactate, carbomers, carboxymethylcellulose sodium, carrageenan, cellulose acetate phthalate, ceratonia, chitosan, copovidone, corn starch, pregelatinized starch, cottonseed oil, dextrates, dextrin, dextrose, ethylcellulose, gelatin, glyceryl behenate, guar gum, hydrogenated vegetable oil, hydroxyethyl cellulose, hydroxypropyl starch, hypromellose, inulin, lactose, liquid glucose, magnesium aluminium silicate, maltodextrin, maltose, methylcellulose, microcrystalline cellulose, pectin, povidone, polaxamer, polycarbophil, polydextrose, polyethylene oxide, polymethacrylates, sodium alginate, stearic acid, sucrose, sunflower oil, tricaprylin, Vitamin E polyethylene glycol succinate, zein, povidone or HPMC, or mixtures thereof. Preferably, the binder is povidone. The binder is suitably present in an amount from about 0.1 to about 30 wt % of the total weight of the formulation. Preferably, the binder is present in an amount from about 1 to about 20 wt % and more preferably in an amount from about 2 to about 15 wt % of the total weight of the formulation.

The formulation may comprise Compound X or a pharmaceutically acceptable salt or solvate thereof in combination with at least one filler and at least one disintegrant, and optionally at least one further excipient. The (or each) further excipient may include a binder, or a lubricant or both.

The formulation may comprise Compound X or a pharmaceutically acceptable salt or solvate thereof in combination with at least one filler and at least one lubricant, and optionally at least one further excipient. The (or each) further excipient may include a binder, or a disintegrant or both.

The formulation may comprise Compound X or a pharmaceutically acceptable salt or solvate thereof in combination with at least one filler and at least one binder, and optionally at least one further excipient. The (or each) further excipient may include a disintegrant, or a lubricant or both.

In each case, the at least one further excipient may include a colouring agent. Suitably, the colouring agent is a polyvinylalcohol-based composition. The colouring agent may be mixed with the excipients and/or mixed with a coating agent (if present). Preferably, the colouring agent is selected from one of the following group: Opadry II 85F33212 orange, Opadry II 85F205017 blue, Opadry II 31K25003 red and Acryl-EZE II 4932180022 white. Suitably the colouring agent is Opadry II 85F205017 blue or Opadry II 31K25003 red. As is well known to those skilled in the art, these are commercial products composed of a series of components: Polyvinyl Alcohol Part. hydrolyzed, Titanium dioxide, Macrogol 3350; Talc, Blue #2/indigo carmine aluminum lake, yellow #6/sunset yellow FCF aluminum lake, Lactose monohydrate, HPMC 2910; Ponceau 4R aluminium lake, triacetin, and Blue #2/indigo carmine aluminum lake, Methacrylic acid copolymer, polaxamer 407, calcium silicate, sodium bicarbonate, sodium lauryl sulfate, red iron oxide, yellow iron oxide, black iron oxide.

The formulation may not be coated. Alternatively, the formulation is coated. If coated, the coating may comprise or consist of the colouring agent.

Suitably, the formulation comprises (optionally consists of) Compound X or a pharmaceutically acceptable salt or solvate thereof in combination with two or three fillers, one binder, one disintegrant, one lubricant, optionally a colouring agent and optionally a coating.

Suitably, the binder is povidone, the disintegrant is sodium croscarmellose and the lubricant is magnesium stearate. Alternatively, the binder is povidone, the disintegrant is crospovidone and the lubricant is magnesium stearate.

The Compound X or a pharmaceutically acceptable salt or solvate thereof used in the formulations may be micronized. Alternatively, the Compound X or a pharmaceutically acceptable salt or solvate thereof used in the formulations may not be micronized. Preferably, the Compound X or a pharmaceutically acceptable salt or solvate thereof used in the formulations is micronized. Advantageously, it has been found that the inclusion of the micronized form of Compound X or a pharmaceutically acceptable salt or solvate thereof in the formulations gives rise to greater uniformity of distribution in the formulations, than with the non-micronized form of Compound X.

When Compound X or a pharmaceutically acceptable salt or solvate thereof is used in micronized form, it may have one or more of the following particle size distribution parameters:
The Dᵥ10 figure for the particles is >0.5 μm; and/or
The Dᵥ50 figure for the particles ranges from around 5 μm to around 150 μm; and/or
The Dᵥ90 figure for the particles is <300 μm.

More preferably, when Compound X or a pharmaceutically acceptable salt or solvate thereof is used in micronized form it may have one or more of the following particle size distribution parameters:
The Dᵥ10 figure for the particles is >1 μm; and/or
The Dᵥ50 figure for the particles ranges from around 10 μm to around 100 μm; and/or
The Dᵥ90 figure for the particles is <2500 μm.

Most preferably, when Compound X or a pharmaceutically acceptable salt or solvate thereof is used in micronized form it may have one or more of the following particle size distribution parameters:
The Dᵥ10 figure for the particles is >2 μm; and/or
The Dᵥ50 figure for the particles ranges from around 20 μm to around 70 μm; and/or
The Dᵥ790 figure for the particles is <180 μm.

When Compound X or a pharmaceutically acceptable salt or solvate thereof is not micronized, it may have one or more of the following particle size distribution parameters:
The Dᵥ10 figure for the particles ranges from around 30 μm to around 150 μm; and/or
The Dᵥ50 figure for the particles ranges from around 200 μm to around 300 μm; and/or
The Dᵥ90 figure for the particles ranges from around 400 μm to around 600 μm.

The amount of Compound X in the formulation will depend on the dosage required. Typically, the amount of Compound X per single formulation will range from about 1 mg to about 1200 mg, preferably from about 5 mg to amount 800 mg, more preferably from about 5 mg to about 400 mg, most preferably from about 5 mg to about 200 mg. The amount of Compound X may be 5 mg, 25 mg, 50 mg, 75 mg, 100 mg, 150 mg, 200 mg, 300 mg, 400 mg, 800 mg or 1200 mg.

Broadly, the pharmaceutical formulation may comprise (by weight of the total formulation excluding any coating present),
from about 0.5 to about 85% of Compound X or a pharmaceutically acceptable salt or solvate thereof, from about 0 to about 98% of filler, from about 0.1 to about 30% of binder, from about 0.1 to about 30% of disintegrant, and from about 0.1 to about 15% of lubricant;
preferably, from about 0.5 to about 85% of Compound X or a pharmaceutically acceptable salt or solvate thereof, from about 5 to about 95% of filler, from about 1 to about 15% of binder, from about 0.1 to about 30% of disintegrant and from about 0.1 to about 15% of lubricant;
more preferably, from about 1 to about 83% of Compound X or a pharmaceutically acceptable salt or solvate thereof, from about 7 to about 90% of filler, from about 2 to about 15% of binder, from about 2 to about 20% of disintegrant, and from about 0.5 to about 8% of lubricant;
most preferably, from about 2 to about 80% of Compound X or a pharmaceutically acceptable salt or solvate thereof, from about 9 to about 87% of filler, from about 3 to about 10% of binder, from about 3 to about 15% of disintegrant, and from about 0.5 to about 5% of lubricant.

Suitably, the ratio of Compound X or a pharmaceutically acceptable salt or solvate thereof to filler (where the filler includes two or more materials, the amount of filler being the total amount of all fillers present) is from about 1:20 to about 10:1 by weight, preferably from about 1:5 to about 5:1, more preferably from about 1:3 to about 3:1, yet more preferably from about 1:2 to about 2:1, and most preferably from about 1:1 by weight.

There is also provided a pharmaceutical formulation comprising Compound X or a pharmaceutically acceptable salt or solvate thereof together with one or more of the following groups of excipients:

A mixture of microcrystalline cellulose and lactose (such as Cellactose® 80—co-processed 75% microcrystalline cellulose and 25% lactose);

### Lactose

Microcrystalline cellulose
Isomalt (such as GalenIQ® 801)
Corn starch (such as Uni Pure FL)
Dibasic dihydrate calcium phosphate (Emcompress®)

### Mannitol

Sodium croscarmellose

### Crospovidone

### Povidone

Magnesium stearate
Opadry II 85F33212 orange
Opadry II 85F205017 blue

### Opadry II 31K25003

Typically, the formulation comprises Compound X or a pharmaceutically acceptable salt or solvate thereof, crospovidone, povidone, magnesium stearate and one or more of the fillers.

There is also provided a pharmaceutical formulation, preferably in the form of a tablet, comprising:
Compound X or a pharmaceutically acceptable salt or solvate thereof;
anhydrous lactose;
microcrystalline cellulose (such as Avicel® PH 101 or MCC 101);
povidone;
sodium croscarmellose;
magnesium stearate; and
colouring agent.

There is also provided a pharmaceutical formulation, preferably in the form of a tablet, comprising:
Compound X or a pharmaceutically acceptable salt or solvate thereof;
pregelatinized starch (such as Starch 1500);
microcrystalline cellulose (such as Avicel® PH 101 or MCC 101);
povidone (such as Povidone K-30);
crospovidone; and
magnesium stearate.

There is also provided a pharmaceutical formulation, preferably in the form of a tablet, comprising:
Compound X or a pharmaceutically acceptable salt or solvate thereof;
mannitol;
microcrystalline cellulose (such as MCC 101);
povidone;
crospovidone; and,
magnesium stearate.

There is also provided a pharmaceutical formulation, preferably in the form of a tablet, comprising:
Compound X or a pharmaceutically acceptable salt or solvate thereof;
microcrystalline cellulose (such as MCC 101);
isomalt (such as isomalt 801);
povidone (such as povidone K-30);
sodium croscarmellose; and
magnesium stearate.

There is also provided a pharmaceutical formulation, preferably in the form of a tablet, comprising:
Compound X or a pharmaceutically acceptable salt or solvate thereof;
microcrystalline cellulose (such as MCC 101);
dibasic dihydrate calcium phosphate;
povidone;
sodium croscarmellose; and
magnesium stearate

Suitable materials to be included in exemplary formulations of the present invention are set out in the tables below.

Composition

Compound X (micronized)
Microcrystalline cellulose (such as MCC 101)
Dibasic dihydrate calcium phosphate (such as Emcompress)
Povidone (such as Povidone K-30)
Sodium croscarmellose
Magnesium stearate
Purified water
coating agent
Purified water coating (ml)*
*Does not appear in the final product.

Composition

Compound X (micronized)
Microcrystalline cellulose (such as MCC 101
Isomalt (such as Isomalt 801)
Povidone (such as Povidone K-30)
Sodium croscarmellose
Magnesium stearate
Purified water
coating agent
Purified water coating (ml)*
*Does not appear in the final product.

Composition

Compound X (micronized)
Microcrystalline cellulose (such as MCC 101)
Lactose (such as Lactose 200M)
Povidone (such as Povidone K-30)
Sodium croscarmellose
Magnesium stearate
Purified water
coating agent
Purified water coating (ml)*
*Does not appear in the final product.

Composition

Compound X (micronized)
Microcrystalline cellulose (such as MCC 101)
Mannitol
Povidone (such as Povidone K-30)
Sodium croscarmellose
Magnesium stearate
Purified water
coating agent
Purified water coating (ml)*
*Does not appear in the final product.

Composition

Compound X (micronized)
Microcrystalline cellulose (such as MCC 101)
Starch 1500
Povidone (such as Povidone K-30)
Sodium croscarmellose
Magnesium stearate
Purified water
coating agent
Purified water coating (ml)*
*Does not appear in the final product.

Composition

Compound X (micronized)
Microcrystalline cellulose (such as MCC 101)
Starch (such as Starch 1500)
Povidone (such as Povidone K-30)
Sodium croscarmellose
Magnesium stearate
Purified water (ml)*
*Does not appear in the final product.

Composition

Compound X (micronized)
Microcrystalline cellulose (such as MCC 101)
Crospovidone
Starch (such as Starch 1500)
Povidone (such as Povidone K-30)
Magnesium stearate
Purified water (ml)*
*Does not appear in the final product.

Composition

Compound X (micronized)
Povidone (such as Povidone K-30)
Microcrystalline cellulose (such as MCC 101)
Talc
Colloidal Hydrated Silica
Starch (such as Starch 1500)
Sodium croscarmellose
Purified water (ml)*
*Does not appear in the final product.

Composition

Compound X (micronized)
Microcrystalline cellulose (such as MCC 101)
Mannitol
Povidone (such as Povidone K-30)
Crospovidone
Magnesium stearate
Purified water (ml)*
*Does not appear in the final product.

Composition

Compound X (micronized)
Microcrystalline cellulose (such as MCC 101)
Isomalt 801
Povidone (such as Povidone K-30)
Sodium croscarmellose
Magnesium stearate
Purified water (ml)*
*Does not appear in the final product.

Composition

Compound X (micronized)
Microcrystalline cellulose (such as MCC 101)
Dibasic dihydrate calcium phosphate (such as Emcompress)
Povidone (such as Povidone K-30)
Sodium croscarmellose
Magnesium stearate
Purified water (ml)*
*Does not appear in the final product.

A preferred formulation is in the form of a tablet and comprises Compound X (preferably micronized Compound X), microcrystalline cellulose (for example MCC 101), mannitol, povidone (for example Povidone K-30), crospovidone and magnesium stearate. The tablet may or may not be coated; preferably the tablet is not coated.

The tablet formulation may consist of the following materials: Compound X (preferably micronized Compound X), microcrystalline cellulose (for example MCC 101), crospovidone, pre-gelatinized starch (for example Starch 1500), povidone (for example Povidone K-30) and magnesium stearate; and purified water as manufacturing adjuvant. The tablet may or may not be coated; preferably the tablet is not coated.

Another preferred formulation is in the form of a tablet and comprises or consists of Compound X (preferably micronized Compound X), microcrystalline cellulose (for example MCC 101), isomalt (for example isomalt 801), povidone (for example Povidone K-30), croscarmellose sodium and magnesium stearate.

Surprisingly, the pharmaceutical formulations disclosed herein exhibit advantageous dissolution characteristics. The dissolution is preferably analysed in accordance with European Pharmacopeia Edition 6, section 2.9.3, paddle apparatus. The paddle apparatus is preferably operated using the following conditions: dissolution volume: 1000 ml (±1%); dissolution medium: (i) HCL 0.01M (pH 2.0±0.05) or (ii) acetate buffer pH 4.5±0.05+0.5%, 0.8%, or 1% Sodium lauryl sulphate; paddle speed: 75 rpm or 100 rpm; time: 45 minutes; and temperature 37±0.5° C. Under these conditions, the formulations may be characterized as exhibiting an average dissolution of at least 50%, suitably at least 65%, preferably at least 70%, more preferably 75%, yet more preferably at least 80%, yet more preferably at least 85%. The formulations may also be characterized as exhibiting an average dissolution of at least 90%. The formulations may also be characterized as exhibiting an average dissolution of at least 95%.

Surprisingly, the pharmaceutical formulations disclosed herein exhibit advantageous disintegration characteristics. The disintegration is preferably analysed in accordance with European Pharmacopeia Edition 6, section 2.9.1. Under these conditions, the formulations may be characterized as exhibiting a disintegration time of less than 30 minutes, suitably less than 25 minutes, preferably less than 20 minutes, more preferably less than 15 minutes, yet more preferably less than 12 minutes, yet more preferably less than 10 minutes. The formulations may also be characterized as exhibiting a disintegration time of less than 8 minutes. The formulations may also be characterized as a disintegration time of less than 6 minutes.

The pharmaceutical formulations of the present invention typically possess a bulk density of at least about 0.5 g/ml, or at least about 0.6 g/ml, such as from about 0.5 to about 0.7 g/ml, including from about 0.55 to about 0.65 g/ml.

The dose of Compound X or a pharmaceutically acceptable salt or solvate thereof may be in the range of about 10 mg/day to about 1500 mg/day, preferably in the range of about 15 mg/day to about 1200 mg/day. The dose of Compound X may be in the range of about 20 mg/day to about 40 mg/day, suitably in the range of about 25 mg/day to about 35 mg/day, typically about 30 mg/day. Alternatively, the dose of Compound X may be in the range of about 100 mg/day to about 1200 mg/day, preferably in the range of about 200 mg/day to about 1200 mg/day. The dose of Compound X may be about 10 mg/day, 15 mg/day, 20 mg/day, 30 mg/day, 40 mg/day, 50 mg/day, 100 mg/day, 200 mg/day, 400 mg/day, 800 mg/day and 1200 mg/day. Preferably, the Compound X is in the form of a single daily dosage. For a required dosage amount of 200 mg/day, one daily dose of a formulation as defined herein including 200 mg Compound X would be appropriate. For a required dosage amount of 400 mg/day, one daily dose of a formulation as defined herein including 400 mg Compound X would be appropriate. For a required dosage amount of 1200 mg/day, one daily dose of a formulation as defined herein including 1200 mg Compound X would be appropriate and so forth. The required dosage may be administered in the form of one or more formulations, for example one or more tablets. For example, if the daily dose is 400 mg, a single formulation (for example a tablet) comprising 400 mg of Compound X (and excipients) may be administered once per day. Alternatively, if the daily dose is 400 mg, two formulations (for example two tablets) each comprising 200 mg of Compound X may be administered. Alternatively, if the daily dose is 400 mg, four formulations (for example four tablets) each containing 100 mg of Compound X may be administered, and so on. As another example, if the daily dose is 1200 mg, a single formulation (for example a tablet) comprising 1200 mg of Compound X (and excipients) may be administered once per day. Alternatively, if the daily dose is 1200 mg, four formulations (for example tablets) each comprising 300 mg of Compound X may be administered. Alternatively, if the daily dose is 1200 mg, twelve formulations (for example tablets) each containing 100 mg of Compound X may be administered, and so on. In a similar manner, other daily doses may be administered in suitable multiples of 5, 25, 50, 100, 200, 400 mg etc.

Preferably the formulation is in unit dosage form, e.g. packaged preparation, the package containing discrete quantities of preparation such as packeted tablets, capsules and powders in vials or ampoules.

The dosages may be varied depending on the requirement of the patient and the severity of the disease. For convenience, the total daily dosage may be divided and administered in portions throughout the day.

Preferably, the formulation is a solid oral dosage form, such as a tablet or a capsule. A tablet formulation may be prepared by direct compression, or by preparing granules comprising compound X and using the granules to prepare the tablet. A capsule may be prepared by preparing granules comprising compound X and using the granules to prepare the capsule. Suitably, each tablet or capsule includes around 5 mg, 25 mg, 50 mg, 75 mg, 100 mg, 150 mg or 200 mg of Compound X.

Optionally, the formulations of the invention include a further pharmaceutically active agent.

The present invention also provides methods of preparing the formulations comprising Compound X as disclosed herein.

In another aspect of the invention, there is provided a method of preparing a pharmaceutical formulation comprising combining, for example, admixing, a therapeutically effective amount of Compound X or a pharmaceutically acceptable salt or solvate thereof:

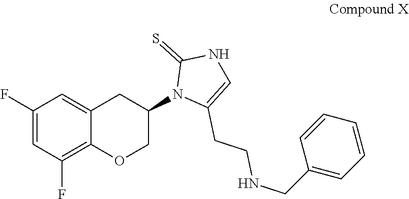 [SMILES: Fc1cc(F)c2c(c1)C[C@@H](n1c(CCNCc3ccccc3)c[nH]c1=S)CO2]

together with one or more pharmaceutically effective excipients.

The methods described herein may be used to make any one of the formulations described herein.

Preferably, the formulation is in the form of a tablet or capsule. The tablet may be prepared by direct compression. Alternatively, the method may involve preparing granules comprising compound X and using the granules to prepare a tablet or a capsule.

There is provided a preferred method of preparing a pharmaceutical formulation, optionally in the form of a tablet, the method comprising:
(a) mixing Compound X or a pharmaceutically acceptable salt or solvate thereof:

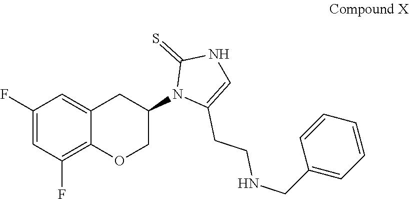 [SMILES: Fc1cc(F)c2c(c1)C[C@@H](n1c(CCNCc3ccccc3)c[nH]c1=S)CO2]

with at least one excipient;
(b) mixing the mixture from step (a) with at least one further excipient;
(c) adding at least one further excipient to the mixture from step (b) and mixing;
(d) adding at least one lubricant to the mixture from step (c) and mixing; and optionally
(e) pressing the mixture from step (d) to form a tablet, optionally with a predetermined weight.

The at least one excipient in step (a) and/or step (b) may be a filler. Suitable fillers include those described above.

Suitably, step (e) is carried out and the pharmaceutical formulation is in the form of a tablet.

There is provided a more preferred method of preparing a pharmaceutical formulation optionally in the form of a tablet, the method comprising:

(a) mixing Compound X or a pharmaceutically acceptable salt or solvate thereof with at least one filler;
(b) mixing the mixture from step (a) with at least one further filler and/or with at least one further excipient;
(c) adding at least one further excipient to the mixture from step (b) and mixing;
(d) adding at least one lubricant to the mixture from step (c) and mixing; and optionally
(e) pressing the mixture from step (d) to form a tablet with a predetermined weight.

Suitable fillers include those described above.

Step (b) may comprise mixing the mixture from step (a) with at least one further filler (such as those described above) and with at least one further excipient. Step (b) may comprise mixing the mixture from step (a) with either the at least one further filler or with the at least one further excipient.

Suitably, step (e) is carried out and the pharmaceutical formulation is in the form of a tablet.

The at least one excipient in step (b) and/or step (c) may be a disintegrant. Suitable disintegrants include those described above.

The at least one excipient in step (b) and/or step (c) may be a binder. Suitable binders include those described above.

As one of skill in the art would know, fillers may also be referred to as diluents. As one of skill in the art would also know, fillers, disintegrants and binders are all excipients.

Preferably, the lubricant is a glidant and/or anti-adherent. Suitable lubricants include those described above.

Compound X may suitably be provided in the form of the hydrochloride salt. However, given the secondary aliphatic amino group, it will be obvious to the skilled technician that other acid salts can be made and are within the scope of the claimed invention.

Compound X may preferably be mixed with excipient(s) in step (a) in a ratio of from about 1:0.5 to about 1:10, more preferably from about 1:1 to about 1:8, more preferably from about 1:2 to about 1:6 and most preferably from about 1:4 to about 1:5.

The mixture from step (a) is preferably mixed with further excipients(s) in step (b) in a ratio of from about 1:0.5 to about 1:10, more preferably from about 1:0.75 to about 1:8, and most preferably about from about 1:3 to about 1:4.

The mixture from step (b) is preferably mixed with the remaining excipients with the exception of the lubricant in step (c).

The excipient(s) in step (a), step (b) and/or step (c) may preferably be a filler selected from those described above.

The excipient(s) in step (b), may more preferably be a filler and comprises mannitol and microcrystalline cellulose. Alternatively, the excipient in step (a), may be a filler and may comprise microcrystalline cellulose, lactose, pre-gelatinized starch, dibasic dihydrate calcium phosphate or isomalt.

The excipient(s) of steps (a) may comprise at least one excipient selected from one or more of the following group: co-processed 75% microcrystalline cellulose and 25% lactose (for example Cellactose® 80 or Microcelac 100, Emcompress® (dibasic dihydrate calcium phosphate), ammonium alginate, compressible sugar, lactose, lactose monohydrate and corn starch (for example StarLac), lactose monohydrate and povidone (for example Ludipress), medium chain triglycerides, talc, tragacanth, Uni Pure FL (corn starch), povidone, sodium croscarmellose, acetyltributyl citrate, acetyltriethyl citrate, alginic acid, aluminium oxide, calcium alginate, calcium carbonate, calcium lactate, dibasic anhydrous calcium phosphate, tribasic calcium phosphate, calcium silicate, calcium sulphate, carbomer, carboxymethylcellulose calcium, carboxymethylcellulose sodium, carrageenan, hydrogenated castor oil, microcrystalline cellulose (such as MCC 101, Avicel® PH 101, Avicel® PH 102), cellulose, silicified microcrystalline cellulose, cellulose acetate, cellulose acetate phthalate, ceratonia, ceresin, chitosan, colloidal silicon dioxide, copovidone, corn starch, pregelatinized starch (for example Starch 1500), croscarmellose sodium, crospovidone, dextrates, dextrin, dextrose, docusate sodium, erythritol, ethylcellulose, fructose, fumaric acid, gelatin, glyceryl behenate, glyceryl monooleate, glyceryl monostearate, glyceryl palmitostearate, glycine, guar gum, hydroxyethyl cellulose, hydroxyethylmethyl cellulose, hydroxypropyl betadex, hydroxypropyl cellulose, hydroxypropyl starch, hypromellose, hypromellose acetate succinate, hypromellose phthalate, inulin, isomalt (for example GaleniQ® 801), kaolin, lactitol, anhydrous lactose, lactose monohydrate, magnesium aluminium silicate, magnesium carbonate, magnesium oxide, maltitol, maltodextrin, maltose, mannitol, methylcellulose, pectin, polacrilin potassium, polaxamer, polycarbophil, polydextrose, poly (DL-lactic acid), polyethylene glycol, polyethylene oxide, polymethacrylates, poly (methyl vinyl ether/maleic anhydride), polyoxyglycerides, polyvinyl acetate phthalate, polyvinyl alcohol, povidone, shellac, simethicone, sodium alginate, sodium chloride, sodium hyaluronate, sodium starch glycolate, sorbitol, starch, pregelatinized starch, sucrose, sugar spheres, sulfobutylether B-cyclodextrin, sunflower oil, titanium dioxide, trehalose, tributyl citrate, triethyl citrate, hydrogenated vegetable oil, vitamin E, polyethylene glycol succinate, microcrystalline wax, white wax, yellow wax, xantham gum, xylitol and zein, and combinations thereof.

Suitably, the excipient is a disintegrant. It is preferred that the disintegrant is added in step (a).

Suitably, the excipient is crospovidone, croscarmellose sodium or sodium starch glycolate.

The mixture from step (c) is suitably mixed with at least one lubricant to produce a composition with a mixture from step (c): lubricant ratio of from about 96:4 to about 99.9:0.1, preferably, from about 98:2 to about 99.5:0.5.

The lubricant(s) in step (d) is/are suitably selected from one or more of the lubricants described above. The preferred lubricant is magnesium stearate.

The further excipients may comprise at least one disintegrant. Suitable disintegrants are described above.

The method may comprise complete addition of the filler(s) and further excipients, except the lubricant, by the completion of step (c).

A colouring agent may be added to the mixture in step (a), step (b) and/or step (c) together with the excipient(s). Suitable colouring agents are described above.

The method may include an additional step of adding a colouring agent to the mixture from step (c) and mixing.

Suitably, the method further comprises film coating the tablet after step (e).

During the coating process, sufficient coating material is used such that the tablets gain around 1% to 15% of their initial weight in coating material; preferably the tablets gain around 3% to 10% of their initial weight in coating material; suitably the tablets gain around 5% to 7% of their initial weight in coating material.

The invention also provides a wet granulation method for preparing a formulation comprising Compound X or a pharmaceutically acceptable salt or solvate thereof. The wet granulation process broadly comprises the following steps:
Pre-mixture
Granulation
Drying
Sieving
Final Blend
Compression

In another aspect of the invention, there is provided a method of preparing a pharmaceutical formulation comprising the following steps:

(a) mixing Compound X or a pharmaceutically acceptable salt or solvate thereof:

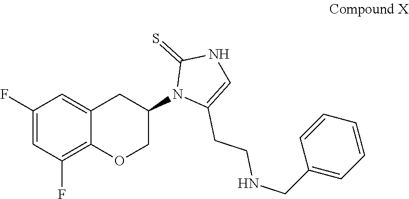 [SMILES: Fc1cc(F)c2c(c1)C[C@@H](n1c(CCNCc3ccccc3)c[nH]c1=S)CO2]

with at least one excipient;
(b) mixing the mixture from step (a) with at least one further excipient;
(c) adding at least one further excipient to the mixture from step (b) and mixing;
(d) adding granulation liquid to the mixture from step (c) and granulating;
(e) drying the granules;
(f) calibrating the granules;
(g) adding at least one lubricant to the granules from step (f) and mixing.

The at least one excipient in step (a) and/or step (b) may be a filler. Suitably both excipients in steps (a) and (b) are fillers, binders, and disintegrants (different to one another).

Suitably, the method further comprises step h) pressing the mixture from step (g) to form a tablet with a predetermined weight.

Alternatively, the method further comprises step h) filling a capsule with a predetermined weight of the mixture from step (g).

Suitably, there is provided a method of preparing a pharmaceutical formulation comprising the following steps:

(a) mixing Compound X or a pharmaceutically acceptable salt or solvate thereof with at least one filler, binder, or disintegrant;
(b) mixing the mixture from step (a) with at least one further filler and/or with at least one further excipient;
(c) adding at least one further excipient to the mixture from step (b) and mixing;
(d) adding granulation liquid to the mixture from step (c) and granulating;
(e) drying the granules;
(f) calibrating the granules;
(g) adding at least one lubricant to the granules from step (f) and mixing; and optionally (h) pressing the mixture from step (g) to form a tablet with a predetermined weight or filling a capsule with a predetermined weight of the mixture from step (g).

The at least one excipient in step (b) and/or step (c) may be a disintegrant. Suitable disintegrants are described above.

The at least one excipient in step (b) and/or step (c) may be a binder. Suitable binders are described above.

Step (b) may comprise mixing the mixture from step (a) with at least one further filler and with at least one further excipient. Alternatively, step (b) comprises mixing the mixture from step (a) with either the at least one further filler or with the at least one further excipient.

Suitably, the tablet is blinded such that the various strengths of the tablet cannot be distinguished.

The wet granulation method according to the invention advantageously allows the granules to be coloured in a homogenous manner in order to give rise to homogenous coloured granules.

Suitably, the at least one further excipient in step (b) and/or (c) comprises a colouring agent. The colouring agent may also be dispersed in the granulation liquid.

Compound X may suitably be provided in the form of the hydrochloride salt.

Suitably, Compound X is mixed with excipient(s) in step (a) in a ratio of 1:0.5 to 1:10, more preferably 1:1 to 1:8, more preferably 1:2 to 1:6, and most preferably 1:4 to 1:5.

The mixture from step (a) is preferably mixed with further excipients(s) in step (b) in a ratio of 1:0.5 to 1:10, more preferably 1:0.75 to 1:8, and most preferably about 1:3 to 1:4.

Suitably the mixture from step (b) is mixed with the remaining excipients with the exception of the lubricant in step (c).

Suitably, the excipient(s) in step (a), step (b) and/or step (c) is a filler selected from one or more of the fillers described above.

Suitably, the excipient(s) in step (a), step (b) and/or step (c) is a filler and comprises microcrystalline cellulose and mannitol. Alternatively, the excipient in step (a), step (b) and/or step (c) is a filler and may comprise Emcompress® (dibasic dihydrate calcium phosphate). Alternatively, the filler is a mixture of microcrystalline cellulose and isomalt. Alternatively, the filler is a mixture of microcrystalline cellulose and pregelatinized starch. Alternatively, the filler is a mixture of microcrystalline cellulose and anhydrous lactose.

Suitably, the excipient(s) of steps (a), step (b) and/or step (c) comprise at least one excipient selected from one or more of the following group: co-processed 75% microcrystalline cellulose and 25% lactose (for example Cellactose® 80 or Microcelac 100, Emcompress® (dibasic dihydrate calcium phosphate), ammonium alginate, compressible sugar, lactose, lactose monohydrate and corn starch (for example StarLac), lactose monohydrate and povidone (for example Ludipress), medium chain triglycerides, talc, tragacanth, Uni Pure FL (corn starch), povidone, sodium croscarmellose, acetyltributyl citrate, acetyltriethyl citrate, alginic acid, aluminium oxide, calcium alginate, calcium carbonate, calcium lactate, dibasic anhydrous calcium phosphate, dibasic anhydrous calcium phosphate, tribasic calcium phosphate, calcium silicate, calcium sulphate, carbomer, carboxymethylcellulose calcium, carboxymethylcellulose sodium, carrageenan, hydrogenated castor oil, microcrystalline cellulose such as MCC 101, Avicel® PH 101, Avicel® PH 102), cellulose, silicified microcrystalline cellulose, cellulose acetate, cellulose acetate phthalate, ceratonia, ceresin, chitosan, colloidal silicon dioxide, copovidone, corn starch, pregelatinized starch, croscarmellose sodium, crospovidone, dextrates, dextrin, dextrose, docusate sodium, erythritol, ethylcellulose, fructose, fumaric acid, gelatin, glyceryl behenate, glyceryl monooleate, glyceryl monostearate, glyceryl palmitostearate, glycine, guar gum, hydroxyethyl cellulose, hydroxyethylmethyl cellulose, hydroxypropyl betadex, hydroxypropyl cellulose, hydroxypropyl starch, hypromellose, hypromellose acetate succinate, hypromellose phthalate, inulin, isomalt, kaolin, lactitol, anhydrous lactose, lactose monohydrate, magnesium aluminium silicate, magnesium carbonate, magnesium oxide, maltitol, maltodextrin, maltose, mannitol, methylcellulose, pectin, polacrilin potassium, polaxamer, polycarbophil, polydextrose, poly (DL-lactic acid), polyethylene glycol, polyethylene oxide, polymethacrylates, poly (methyl vinyl ether/maleic anhydride), polyoxyglycerides, polyvinyl acetate phthalate, polyvinyl alcohol, povidone, shellac, simethicone, sodium alginate, sodium chloride, sodium hyaluronate, sodium starch glycolate, sorbitol, starch, pregelatinized starch, sucrose, sugar spheres, sulfobutylether B-cyclodextrin, sunflower oil, titanium dioxide, trehalose, tributyl citrate, triethyl citrate, hydrogenated vegetable oil, vitamin E, polyethylene glycol succinate, microcrystalline wax, white wax, yellow wax, xantham gum, xylitol and zein, and combinations thereof.

The most preferred excipients to be added in step (a) and/or step (c) are binder(s) or disintegrant(s). Suitably, binder(s) or disintegrant(s) are only added in step (c), i.e., not in any other steps.

The granulation liquid may be water, an alcohol such as ethanol or isopropanol, propylene carbonate and/or acetone. Suitably the granulation liquid is water, and more preferably purified water (i.e. purified water European Pharmacopoeia 6^{th} Edition (EP6)/United States Pharmacopoeia 33 (USP33)).

The granules are preferably dried until the loss on drying (LOD) is less than or equal to 10.0%, preferably from 1 to 4.0%. Preferably the dried granules are calibrated through a 0.5 to 2.5 mm sieve, preferably through a 0.8 to 1.5 mm sieve. Loss on drying is preferably calculated using the method described in the experimental section below.

The granules from step (f) may be mixed with at least one lubricant to produce a composition with a granules:lubricant ratio of from about 96:4 to about 99.9:0.1, preferably from about 98:2 to about 99.5:0.5.

The lubricant(s) in step (g) is/are suitably selected from one or more of the following lubricants described above. Preferably, the lubricant is a glidant. Preferably, the lubricant is magnesium stearate.

Suitably, the colouring agent may be added with the excipient(s) in step (a), (b) and/or (c). The colouring agent is preferably added in step (a) or (b) to allow a better colouring agent distribution and homogeneity.

Alternatively, the colouring agent may be dispersed in the granulation liquid.

The amount of colouring agent may be from 1-50% by weight of the total formulation (excluding any coating present), more preferably 3-25%, more preferably 8-20%, more preferably 12-16% and most preferably about 13-14%.

Suitably, the method further comprises film coating the tablet after step (h).

During the coating process, sufficient coating material is used such that the tablets gain around 1% to 15% of their initial weight in coating material; preferably the tablets gain around 3% to 10% of their initial weight in coating material; suitably the tablets gain around 5% to 7% of their initial weight in coating material.

Preferably, the method includes an additional step of mixing the at least one filler with an active ingredient prior to step (a).

As described above, the formulation may be a capsule or tablet. Most preferably the formulation is a tablet. The tablets may be any appropriate shape as would be known to one of skill in the art. Suitably the tablets are circular, oblong, oval circular or oval shaped or have any other adequate shape, and preferably the tablets are oblong shaped.

All of the methods described above may also include the addition of a further pharmaceutically active agent to the formulation. The further pharmaceutically active agent may be added in a separate step or may be added during one of the steps previously described.

The dosages of the tablet prepared in accordance with the present invention may be varied depending on the requirement of the patient and the severity of the disease. The formulation of the present invention may also comprise at least one other pharmaceutically active ingredient. This pharmaceutically active ingredient may be added to the formulation during any of the method steps described above, or it may be added in a separate step.

As described above, the amount of Compound X or a pharmaceutically acceptable salt or solvate thereof per single formulation will range from about 1 mg to about 400 mg, preferably from about 2 mg to amount 300 mg, more preferably from about 3 mg to about 300 mg, most preferably from about 5 mg to about 200 mg. The amount of Compound X or a pharmaceutically acceptable salt or solvate thereof may be 5 mg, 25 mg, 100 mg, 200 mg, 300 mg or 400 mg. Preferred amounts of Compound X or a pharmaceutically acceptable salt or solvate thereof are 5 mg, 25 mg, 100 mg and 200 mg per single oral dosage form.

A formulation disclosed herein may be used to treat disorders where a reduction in the hydroxylation of dopamine to noradrenaline is of therapeutic benefit, or for inhibiting Dopamine Beta Hydroxylase (DβH). Such disorders are disclosed in WO 2008/136695 and WO2014/077715.

Formulations comprising Compound X or a pharmaceutically acceptable salt or solvate thereof and prepared in accordance with the present invention are also appropriate for use in treating pulmonary arterial hypertension by administering a formulation comprising a therapeutically effective amount of Compound X or a pharmaceutically acceptable salt or solvate thereof as described above to a patient in need thereof. In accordance with the present invention there is also provided a method of treating pulmonary arterial hypertension which comprises administering to a patient in need thereof a therapeutically effective amount of a pharmaceutical formulation comprising Compound X or a pharmaceutically acceptable salt or solvate thereof.

Compound X may be combined with one or more active pharmaceutical ingredients and used in treating pulmonary arterial hypertension. The at least one other active pharmaceutical ingredient may be one or more selected from the following list: epoprostenol, iloprost, bosentan, ambrisentan, sitaxentan, sildenafil, tadalafil, amlodipine, felodipine, diltiazem, nifedipine, nicardipine isosorbide dinitrate, isosorbide-5-mononitrate, warfarin, captopril, enalapril, lisinopril, benazepril, fosinopril, trandolapril, quinapril, ramipril, perindopril, zofenopril, cilazapril, imidapril, losartan, candersartan, olmesartan, irbesartan, eprosartan, telmisartan, valsartan, acetazolamide, dichlorphenamide, methazolamide, furosemide, ethacrynic acid, torasemide (torsemide), azosemide (axosemide), piretanide, tripamide, hydrochlorothiazide, chlorothiazide, bendroflumethiazide, methyclothiazide, polythiazide, trichlormethiazide, chlorthalidone, indapamide, metolazone, quinethazone, amiloride, triamterene, spironolactone, canrenone, potassium canrenoate, macitentan, riociguat, treprostinil, epoprostenol, and eplerenone. A preferred further active pharmaceutical ingredient is bosentan.

The formulation may be administered with other medications as a combination therapy. The combination of active substances may be administered simultaneously, sequentially or separately in accordance with the present invention.

Examples of formulations in accordance with the present invention are provided in the following tables.

| | Strength | | |
|---|---|---|---|
| | 5 mg | 25 mg | 100 mg |
| | Composition | | |
| | I | II | III |
| Starting material | Quantity (mg/tablet) | | |
| Compound X | 5 | 25 | 100 |
| Microcrystalline cellulose (such as | 100.75 | 90.75 | 53.25 |
| Avicel PH 101) | | | |
| Lactose (such as Lactose 200M) | 100.50 | 90.50 | 53.00 |
| Povidone (such as Povidone K-30) | 13.75 | 13.75 | 13.75 |
| Sodium Croscarmellose | 13.75 | 13.75 | 13.75 |
| Magnesium Stearate | 2.75 | 2.75 | 2.75 |
| Opadry II 85F205017 blue | 38.50 | 38.50 | 38.50 |

| Composition IV | Unit quantity (mg) |
|---|---|
| Compound X (micronized) | 100.00 |
| Microcrystalline cellulose (such as | 54.75 |
| MCC 101) | |
| Dibasic dihydrate calcium phosphate | 54.40 |
| (such as Emcompress) | |
| Povidone K-30 | 11.75 |
| Sodium croscarmellose | 11.75 |
| Magnesium stearate | 2.35 |
| Purified water | 0.05 |
| Acryl-EZE II 493Z180022 white | 6% weight gain** |
| Purified water coating (ml) | q ad for 20% solution |
| Total | 235.00 |

**during the coating process the tablets gain 6% of their initial weight in coating material

| Composition V | Unit quantity (mg) |
|---|---|
| Compound X (micronized) | 100.00 |
| Microcrystalline cellulose (such as | 54.75 |
| MCC 101) | |
| Isomalt (such as Isomalt 801) | 54.40 |
| Povidone K-30 | 11.75 |
| Sodium croscarmellose | 11.75 |
| Magnesium stearate | 2.35 |
| Purified water | 0.05 |
| Acryl-EZE II 493Z180022 white | 6% weight gain** |
| Purified water coating (ml) | q ad for 20% solution |
| Total | 235.00 |

**during the coating process the tablets gain 6% of their initial weight in coating material

| Composition VI | Unit quantity (mg) |
|---|---|
| Compound X (micronized) | 100.00 |
| Microcrystalline cellulose (such as | 54.75 |
| MCC 101) | |
| Lactose (such as Lactose 200M) | 54.40 |
| Povidone K-30 | 11.75 |
| Sodium croscarmellose | 11.75 |
| Magnesium stearate | 2.35 |
| Purified water | 0.05 |
| Acryl-EZE II 493Z180022 white | 6% weight gain** |
| Purified water coating (ml) | q ad for 20% solution |
| Total | 235.00 |

**during the coating process the tablets gain 6% of their initial weight in coating material

| Composition VII | Unit quantity (mg) |
|---|---|
| Compound X (micronized) | 100.00 |
| Microcrystalline cellulose (such as | 54.75 |
| MCC 101) | |
| Mannitol | 54.40 |
| Povidone K-30 | 11.75 |
| Sodium croscarmellose | 11.75 |
| Magnesium stearate | 2.35 |
| Purified water | 0.05 |
| Acryl-EZE II 493Z180022 white | 6% weight gain** |
| Purified water coating (ml) | q ad for 20% solution |
| Total | 235.00 |

**during the coating process the tablets gain 6% of their initial weight in coating material

| Composition VIII | Unit quantity (mg) |
|---|---|
| Compound X (micronized) | 100.00 |
| Microcrystalline cellulose (such as | 54.75 |
| MCC 101) | |
| Starch (such as Starch 1500) | 54.40 |
| Povidone K-30 | 11.75 |
| Sodium croscarmellose | 11.75 |
| Magnesium stearate | 2.35 |
| Purified water | 0.05 |
| Acryl-EZE II 493Z180022 white | 6% weight gain** |
| Purified water coating (ml) | q ad for 20% solution |
| Total | 235.00 |

**during the coating process the tablets gain 6% of their initial weight in coating material

| Composition IX | Unit quantity (mg) |
|---|---|
| Compound X (micronized) | 200.00 |
| Microcrystalline cellulose (such as MCC 101) | 109.50 |
| Starch (such as Starch 1500) | 108.80 |
| Povidone K-30 | 23.50 |
| Sodium croscarmellose | 23.50 |
| Magnesium stearate | 4.7 |
| Purified water | 0.05 |
| Total | 470.00 |

| Composition X | Unit quantity (mg) |
|---|---|
| Compound X (micronized) | 200.00 |
| Microcrystalline cellulose (such as MCC 101) | 109.50 |
| Talc | 5.36 |
| Colloidal Hydrated Silica | 5.36 |
| Starch (such as Starch 1500) | 108.80 |
| Povidone (such as Povidone K-30) | 23.50 |
| Sodium croscarmellose | 23.50 |
| Purified water | 0.05 |
| Total | 476.02 |

| | Strength | | |
|---|---|---|---|
| | 50 mg | 100 mg | 200 mg |
| | Composition | | |
| | XI | XII | XIII |
| Starting material | Quantity (mg/tablet) | | |
| Compound X | 50 | 100 | 200 |
| Microcrystalline cellulose (such as | 184.7 | 159.7 | 109.5 |
| MCC 101) | | | |
| Pre-gelatinied starch | 183.6 | 158.6 | 108.8 |
| Povidone (such as Povidone K-30) | 23.5 | 23.5 | 23.5 |
| Crospovidone | 23.5 | 23.5 | 23.5 |
| Magnesium stearate | 4.7 | 4.7 | 4.7 |
| Purified water (ml) | — | — | — |
| Total | 470 | 470 | 470 |

| | Strength | | | |
|---|---|---|---|---|
| | 5 mg | 25 mg | 100 mg | 200 mg |
| | Composition | | | |
| | XIV | XV | XVI | XVII |
| Starting material | Quantity (mg/tablet) | | | |
| Compound X | 5 | 25 | 100 | 200 |
| Microcrystalline cellulose (such as | 108.75 | 98.75 | 61.25 | 11.25 |
| MCC 101) | | | | |
| Mannitol | 108.75 | 98.75 | 61.25 | 11.25 |
| Povidone (such as Povidone K-30) | 12.5 | 12.5 | 12.5 | 12.5 |
| Crospovidone | 12.5 | 12.5 | 12.5 | 12.5 |
| Magnesium stearate | 2.5 | 2.5 | 2.5 | 2.5 |
| Purified water (ml) | — | — | — | — |
| Total | 250 | 250 | 250 | 250 |

According to a further aspect of the present invention, there is provided a method of preparing Compound X, the individual (R)- or (S)-enantiomer of Compound X, or a mixture of the (R)- and (S)-enantiomer of Compound X; or salt thereof, which process comprises the following steps (i)-(iii):

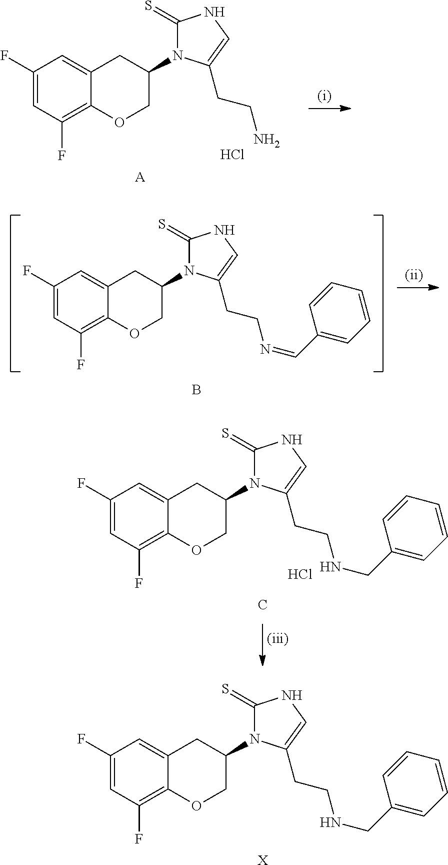

Suitable reagents for use in this process are as follows:

Step (i): benzaldehyde, tetrahydrofuran (THF), triethylamine, sodium sulfate
Step (ii): (a) isopropranol, sodium borohydride; (b) hydrochloric acid (HCl)
Step (iii): sodium hydroxide, methanol/water mixture.

Suitable salts of Compound X include the L-tartrate, hydrochloride, mesylate, tosylate, trifluoroacetate, citrate, glycolate, oxalate and acetate salts. The preferred salt is the L-tartrate salt. Suitably, the compound of Formula X is prepared either in free base form or as the L-tartrate salt thereof. Preferably, the compound of Formula X is prepared as the L-tartrate salt thereof.

The compound of Formula A may be prepared using any process disclosed herein or any process known in the prior art, for example using a process disclosed in WO 2004/033447.

Synthetic Method

### 1. Step (i)

Under an argon atmosphere a suspension of a compound of Formula A (10 g, 28.8 mmol, 1.00 eq) and sodium sulphate (10 g, 70.4 mmol, 2.45 eq) in THF (60 mL, 6 Vol) was stirred at 20° C. Benzaldehyde (3.07 mL, 30.2 mmol, 1.05 eq) was added at 20° C. and the dropping funnel was rinsed with THF (10 mL, 1 Vol). Triethylamine (4.82 mL, 34.6 mmol, 1.20 eq) was added dropwise at 23° C. for 30 minutes. The dropping funnel was rinsed with THF (10 mL, 1 Vol). The suspension was stirred at 23° C. for 4 hours and then filtered. The white solid was washed with THF (30 mL, 3 Vol) and the filtrate was concentrated to ˜2 Vol THF. The yellow solution of the imine in THF was kept under argon.

### 2. Step (ii)

The obtained solution of a compound of Formula B in THF (2.0 Vol) was added dropwise to a 0° C. cold solution of sodium borohydride (1.31 g, 0.03 mol, 1.20 eq) in THF (20 mL, 2 Vol) and 2-propanol (2.65 mL, 1.20 mol eq) over 1 hour. The dropping funnel was washed with THF (2.5 mL). The suspension was stirred at 0° C. for 1 hour and then at 23° C. for 14 hours. After dropwise addition of a mixture of 2-propanol (47.4 mL, 4.7 Vol) and water (5 mL, 0.5 Vol) at 23° C. over 1 hour, the suspension was heated within 1 hour to 65° C. and refluxed at 65° C. for 7 hours. The suspension was cooled to 20° C. A solution of 4M HCl (45 mL, 4.5 Vol) was added dropwise over 1 hour. The suspension was stirred at 20° C. for 15 h. The precipitate was filtered, washed with water (4 Vol), HCl (4M, 2 Vol), water (2 Vol) and 2-propanol (2 Vol). The compound of Formula C (white product, 9.95 g, 79%) was dried at 30° C. for 1 day.

3. Step (iii)—Method 1

Under an argon atmosphere, the compound of Formula C (4.0 g, 9.13 mmol, 1.00 eq) was added to methanol (56.4 mL, 14.1 V) and heated to 45° C. The mixture was stirred until an almost clear solution is formed. The solution was then filtered and the filter washed with methanol (4 mL, 1V). Water (9.3 mL, 2.3V) was added to the solution. The suspension was heated to 65° C. and sodium hydroxide solution (1 M, 2.5 eq) was added over 75 minutes. A white solid precipitated and stirring continued at 65° C. for 45 minutes. The pH value was measured as pH=>8. The suspension was cooled to 23° C. over 100 minutes and stirred at 23° C. for 45 minutes. The white solid was filtered and washed with a mixture of methanol and water (4V, 1:1). The resulting product, Compound X product was then dried at 50° C. for 2 days.

4. Step (iii)—Method 2

Under an argon atmosphere, the compound of Formula C (352 g, 1.00 eq) was added to methanol (10 V) and heated to 45° C. The mixture was stirred until an almost clear solution is formed. The solution was filtered and the filter washed with methanol (4V). The solution was transferred into the reactor and the flask washed with methanol (1 V). Water (2.3V) was added to the solution at 45° C. over 15 minutes. The suspension was heated to 65° C. and sodium hydroxide solution (1 M, 2.5 eq) was added over 75 minutes. A white solid precipitated and stirring continued at 65° C. for 45 minutes. The suspension was cooled to 20° C. over 100 minutes and stirred at 20° C. for 45 minutes. The white solid was filtered and washed with a mixture of methanol and water (4V, 1:1). The resulting product, Compound X (263 g, 81.4%), was the dried at 50° C. for 3 days.

### EXPERIMENTAL STUDIES

### Equipment

Experimental studies were performed using the following commercially available equipment:
Balance Mettler Toledo model PM 1200
Balance AND GX-1000
Mixer/Granulator Diosna P 1/6
Fluid Bed Dryer Diosna Minilab XP
Oscillating 1.0 mm sieve coupled to Erweka KU1
V blender coupled to Erweka rotor type AR402
Killian Tablet Press
Fette 1200iC Rotary Tablet Press

In addition, the following equipment was used to test the samples:
Balance Mettler Toledo, model AG 245
Waters Alliance HPLC, model 2690, with a diode array detector model 996

### Parameters and Methods

The direct compression tablets batches were prepared using the following procedure:
1. Compound X was mixed with the first filler in a 1:4 proportion (10 minutes, 25 RPM, V blender).
2. The blend from the previous step was mixed with the second filler in a proportion of 1:1 (10 minutes, 25 RPM, V blender).
3. The remaining excipients with the exception of the lubricant were added and the blend and mixed (10 minutes, 25 RPM, V blender).
4. The lubricant was added to the blend from the previous step and mixed (5 minutes, 25 RPM, V blender).
5. A tablet with the previously defined target weight was formed by compression (using an oblong punch).

The wet granulation batches were prepared using the following procedure:
1. Compound X was mixed with the first filler in a 1:4 proportion (High Shear Mixer/Granulator, 3 minutes).
2. The blend from the previous step was mixed with the second filler in a proportion of 1:1 (High Shear Mixer/Granulator, 3 minutes).
3. The remaining excipients with the exception of the lubricant were added to the blend from the previous step and mixed (High Shear Mixer/Granulator, 3 minutes).
4. The granulation liquid was added and the resultant mixture granulated to produce granules (in a High Shear Mixer/Granulator).
5. The granules were dried until the LOD is less than 3.0% (in a Fluid Bed Dryer). The LOD was measured in an infratester machine following the method given in the European Pharmacopeia 6^{th} Edition, Chapter 2.2.32.
6. The dried granules were calibrated (through a 1.0 mm sieve).
7. The lubricant was added to the granules and mixed (V blender, 5 minutes, 25 RPM)
8. A tablet with the previously defined target weight was formed by compression (using an oblong punch).

In the wet granulation tests, in some cases the colouring agent was added in step 1 or 2, and in other cases it was added with the granulation liquid.

The wet granulation manufacture of all the granules was performed in a laboratorial high shear granulator and a laboratorial fluid bed dryer. The final blend (i.e. from step 7) was performed in a laboratorial cubic blender and the compression in a laboratorial eccentric machine.

The wet granulation process had a high pre-mixture time in order to achieve good colouring agent homogeneity. Overall the mixture took 9 minutes for steps 1, 2 and 3.

API assay The Compound X assay was performed using HPLC as described herein.

### Analytical Procedures

The pharmaceutical formulations of the present invention may be prepared and analysed according to the methods disclosed herein using equipment that is commercially available. Pharmaceutical formulations prepared according to the present invention may also be analysed using methods known in the art, for example as disclosed in the European and United States Pharmacopeias (e.g. European Pharmacopeia 6^{th} Edition and United States Pharmacopeia 33).

### 1. Dissolution

The analytical conditions adopted for dissolution testing of Compound X tablets are summarized below.

### Condition Set (i)

1. Rotating paddle apparatus (European Pharmacopeia Edition 6, section
2.9.3, paddle apparatus)

| | |
|---|---|
| 2. Dissolution medium | HCl 0.01 M solution, pH 2.00 ± 0.05 |
| 3. Volume | 1000 ml (±1%) |
| 4. Temperature | 37.0 ± 0.5° C. |
| 5. Stirrer speed | 100 ± 4 rpm for 5 mg tablets |
| | 75 ± 3 rpm for 25 mg and 100 mg tablets |

### Condition Set (ii)

1. Rotating paddle apparatus (European Pharmacopeia Edition 6, section
2.9.3, paddle apparatus)

| | |
|---|---|
| 2. Dissolution medium | acetate buffer pH 4.5 + 0.05 + 1% Sodium |
| | lauryl sulphate |
| 3. Volume | 1000 ml (±1%) |
| 4. Temperature | 37.0 + 0.5° C. |
| 5. Stirrer speed | 100 ± 4 rpm for 100 mg tablets and |
| | 200 mg tablets |
| | 75 ± 3 rpm for 50 mg tablets |

Condition Set (iii)

1. Rotating paddle apparatus (European Pharmacopeia Edition 6, section
2.9.3, paddle apparatus)

| | |
|---|---|
| 2. Dissolution medium | acetate buffer pH 4.5 + 0.05 + 0.5% Sodium |
| | lauryl sulphate |
| 3. Volume | 1000 ml (±1%) |
| 4. Temperature | 37.0 + 0.5° C. |
| 5. Stirrer speed | 75 ± 3 rpm |

### Condition Set (iv)

1. Rotating paddle apparatus (European Pharmacopeia Edition 6, section
2.9.3, paddle apparatus)

| | |
|---|---|
| 2. Dissolution medium | acetate buffer pH 4.5 + 0.5% Sodium |
| | lauryl sulphate |
| 3. Volume | 1000 ml (±1%) |
| 4. Temperature | 37.0 ± 0.5° C. |
| 5. Stirrer speed | 100 rpm ± 3 rpm |

### 2. Water Content

The water content in Compound X drug was determined by volumetric KF (Karl Fischer) titration.

### 3. Assay (HPLC)

Drug product assay, enantiomeric purity and degradation products assay were determined by HPLC.

### 4. Particle Size Distribution

The particle size distribution of Compound X was determined by laser light diffraction. Particle size determination experiments were performed in a Malvern Mastersizer 2000 laser difractometer equipped with a wet dispersion unit. The analytical information obtained was acquired and processed with the software Malvern Mastersizer 5.54.

The particle size distribution was measured on samples both before micronization and after micronization.

A volume weighted distribution was obtained for the samples. The contribution of each particle in the distribution relates to the volume of that particle, i.e. the relative contribution will be proportional to (size)³.

The parameters (DᵥX) are reported below based on the maximum particle size for a given percentage volume of the sample. In DᵥX, D stands for diameter, v indicates a volume distribution weighting, and X is the percentage of sample below this particle size. For example, the Dᵥ50 would be the maximum particle diameter below which 50% of the sample volume exists.

### Experimental Results

### Compression Force and Punch Shape

The effect of compression force and punch shape on formulation characteristics was investigated.

### Round Punch 10 RPM Compression Force Evaluation:

| | | | | | Disinte- |
|---|---|---|---|---|---|
| Compression | Average | Thickness | Hardness | Friability | gration |
| force (KN) | weight (mg) | (mm) | (N) | (%) | time (s) |
| 6 | 302 | 3.43 | 25 | 0.3 | 68 |
| 16 | 305 | 3.04 | 79 | 0.05 | 297 |
| 24 | 301 | 2.93 | 98 | 0.05 | 393 |

### Oblong Punch 10 RPM Compression Force Tablet Evaluation:

| | | | | | Disinte- |
|---|---|---|---|---|---|
| Compression | Average | Thickness | Hardness | Friability | gration |
| force (KN) | weight (mg) | (mm) | (N) | (%) | time (s) |
| 8.8 | 300 | 5.12 | 173 | 0.03 | 434 |
| 16.4 | 302 | 4.96 | 233 | 0.13 | 630 |
| 27 | 304 | 4.93 | 257 | 0.1 | 725 |

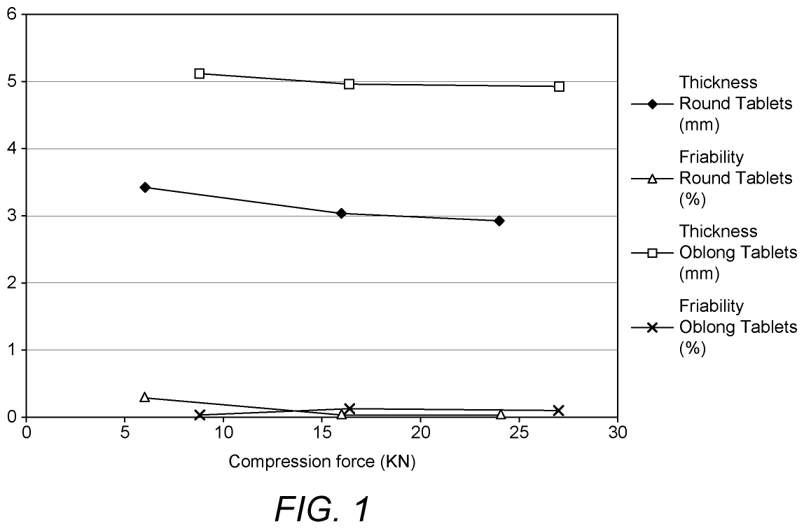

FIG. 1 shows the evaluation of oblong tablet versus round tablet compression parameters (thickness and friability) at a machine speed of 10 RPM.

From FIG. 1 it can be seen that the increase in compression force has no influence on the friability values for both shapes, even though at lower compression forces the friability values in the round tablet shape tend to increase. Regarding the thickness values, they are always lower in the round shape, and in both shapes they decrease with the increase of the compression force, but the decrease is more notable in the round shape tablets.

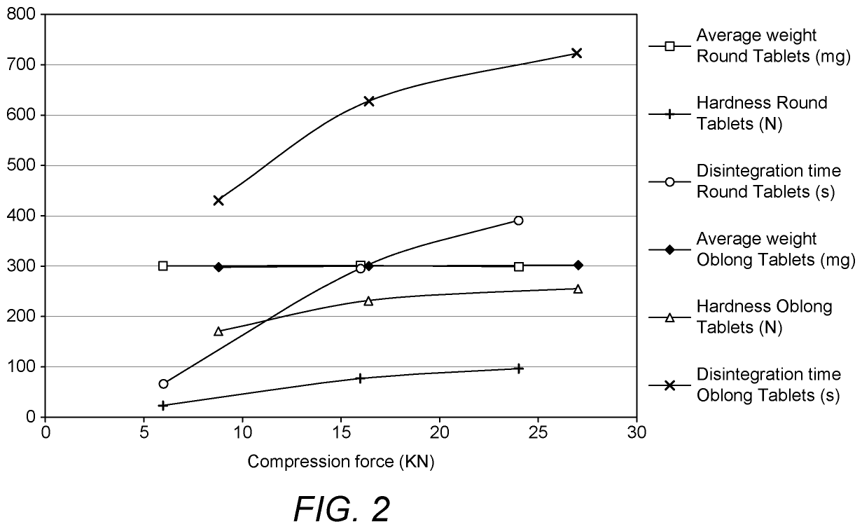

FIG. 2 shows the evaluation of oblong tablet versus round tablet compression parameters (average weight, hardness and disintegration time) at a machine speed of 10 RPM.

From FIG. 2 it can be seen that the average weight in both tablet shapes is not influenced by changes in the compression force. It is also evident that the oblong shape tablets present higher hardness values relative to the round shape tablets in all tested compression forces. In both tablet shapes, it is also evident that when the compression force is increased there is a small increase in the hardness followed by stabilization; this indicates that the compressed formulation material suffers a plastic deformation. The disintegration time is higher in the oblong shape tablets, probably due to its higher hardness, but the higher values are still below 15 minutes (900 s). In both tablet shapes, there is an increase of the disintegration time aligned with increase in the compression force.

An oblong shape tablet was chosen for the further development of the formulations for Compound X due to preferred hardness values.

The speed of the compression machine was varied to determine the effect on tablet parameters. This was a test in which tablets were manufactured at different machine rotation speeds. This test provides information about the compressibility characteristics of the powder/granules with the machine speed variation and helps to predict problems when the mixture is compressed on an industrial scale.

### Oblong Punch Speed Challenge Results:

| | Average | Thickness | Hardness | Friability | Disintegration |
|---|---|---|---|---|---|
| RPM | weight (mg) | (mm) | (N) | (%) | time (s) |
| 10 | 302 | 4.96 | 233 | 0.13 | 630 |
| 20 | 304 | 4.99 | 232 | 0.1 | 658 |
| 30 | 304 | 4.99 | 221 | 0.16 | 695 |
| 40 | 303 | 4.99 | 206 | 0.1 | 655 |

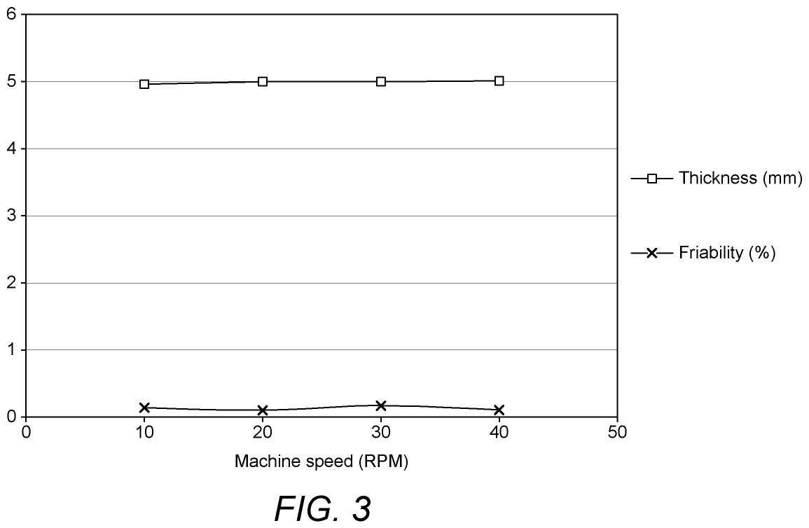

FIG. 3 shows the speed challenge oblong tablet compression parameters evaluation (thickness and friability) at a compression force of 17 KN.

An increase in the machine speed (10-40 RPM) had no impact in the thickness and friability of the resulting tablets.

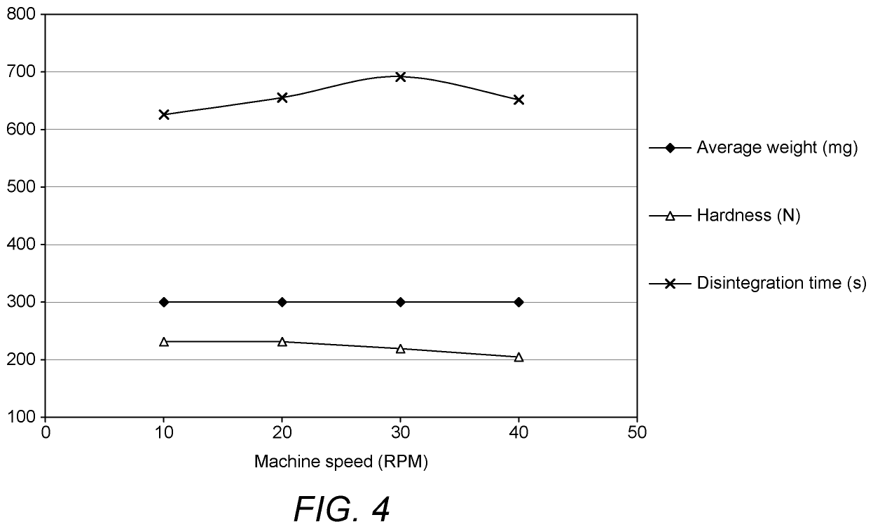

FIG. 4 shows the speed challenge oblong tablet compression parameters evaluation (average weight, hardness and disintegration time) at a compression force of 17 KN.

The increase in the machine speed also had no influence on the average weight, which indicates that the granules have good flow characteristics. However, an increase in the machine speed influenced the hardness values, and for that reason a specific compression time was needed to give a good hardness. Based on this, it was predicted that if the speed was further increased the hardness values would decrease more. These results show that the formulation is suitable for industrial scale production, as this test was done in an industrial type machine.

### Dissolution Studies

Dissolution testing was undertaken for a variety of formulations comprising Compound X in accordance with the present invention.

### Dissolution Results for Tablet Batches Containing 5 mg, 25 mg and 100 mg of Compound X:

| | | | | | | | API |
|---|---|---|---|---|---|---|---|
| | | | | | | | (100 mg |
| | 5 mg | | 25 mg | | 100 mg | | drug in |
| Time | Batch | Batch | Batch | Batch | Batch | Batch | capsule) |
| (min) | 36 | 40 | 37 | 41 | 31 | 42 | N/A |
| 15 | NP | NP | 88% | 85% | 82% | 73% | 3.5% |
| 30 | 103% | NP | 95% | 94% | 89% | 98% | 5.6% |
| 45 | NP | 94% | 100% | 99% | 93% | 99% | 6.9% |

NP—not performed

In addition to Compound X, Batches 31, 36, 37, 40, 41 and 42 contained the following excipients: lactose, microcrystalline cellulose, croscarmellose sodium, povidone, magnesium stearate and a colouring agent.

The results given above indicate that the wet granulation formulation developed for all Compound X dosages is able to successfully release the API as the dissolution percentages are above 89% at the end of 30 minutes in all the presented batches. These dissolution data were surprisingly higher than the observed for the pure API inside a capsule (DIC), showing that developed composition was able to improve more than 10-fold the dissolution of the API.

### Analysis of Selected Formulations

The following formulations were prepared on a lab/pilot scale by a wet granulation method. All formulations included a coating comprising the colorant Opadry II 85F205017 blue.

Composition of the active product (Compound X tablets, 5 mg, 25 mg and 100 mg):

| | Formulation | | | |
|---|---|---|---|---|
| | I | II | III | |
| | (Batch 40) | (Batch 41) | (Batch 42) | |
| | Strength | | | |
| | 5 mg | 25 mg | 100 mg | |
| Starting material | Quantity (mg/tablet) | | | Function |
| Compound X | 5 | 25 | 100 | Active |
| | | | | substance |
| Avicel PH 101 | 100.75 | 90.75 | 53.25 | Diluent |
| Lactose 200M | 100.50 | 90.50 | 53.00 | Diluent |
| Povidone K-30 | 13.75 | 13.75 | 13.75 | Binder |
| Sodium | 13.75 | 13.75 | 13.75 | Disintegrant |
| Croscarmellose | | | | |
| Magnesium Stearate | 2.75 | 2.75 | 2.75 | Glidant |
| Opadry II | 38.50 | 38.50 | 38.5 | Colorant |
| 85F205017 blue | | | | |
| Purified water* | 60 μl | 58 μl | 52 μl | Granulation |
| | | | | liquid |

*Does not appear in the final product.

### Comparative Example

| | Formulation | |
|---|---|---|
| | Strength | |
| | placebo | |
| Starting material | Quantity (mg/tablet) | Function |
| Avicel PH 101 | 100.50 | Diluent |
| Lactose 200M | 100.25 | Diluent |
| Povidone K-30 | 13.75 | Binder |
| Sodium | 13.75 | Disintegrant |
| Croscarmellose | | |
| Magnesium Stearate | 5.50 | Glidant |
| Opadry II | 41.25 | Colorant |
| 85F205017 blue | | |
| Purified water* | 68 μl | Granulation liquid |

The formulations were prepared according to the wet granulation process described above with the colouring agent added during step (a) or step (b) of the wet granulation method, or was added with the granulating agent.

### Batch Formula

Batch formula for Compound X tablets, 5 mg, 25 mg and 100 mg

Typical batch size: 5000 tablets (other batch sizes may be manufactured)

| | Formulation | | | |
|---|---|---|---|---|
| | A | B | C | |
| | (Batch 40) | (Batch 41) | (Batch 42) | |
| | Strength | | | |
| | 5 mg | 25 mg | 100 mg | |
| Starting material | Quantity (g/batch) | | | Function |
| Compound X | 25 | 125 | 500 | Active |
| | | | | substance |
| Avicel PH 101 | 503.75 | 453.75 | 266.25 | Diluent |
| Lactose 200M | 502.50 | 452.50 | 265.00 | Diluent |
| Povidone K-30 | 68.75 | 68.75 | 68.75 | Binder |
| Sodium | 68.75 | 68.75 | 68.75 | Disintegrant |
| Croscarmellose | | | | |
| Magnesium Stearate | 13.75 | 13.75 | 13.75 | Glidant |
| Opadry II | 192.50 | 192.50 | 192.50 | Colorant |
| 85F205017 blue | | | | |
| Purified water* | 300 ml | 290 ml | 260 ml | Granulation |
| | | | | liquid |

*Does not appear in the final product.

Batch formula for Compound X capsules, 5 mg, 25 mg and 100 mg

Typical batch size: 5000 tablets (other batch sizes may be manufactured)

| | Formulation | |
|---|---|---|
| | Strength | |
| | placebo | |
| Starting material | Quantity (g/batch) | Function |
| Avicel PH 101 | 502.50 | Diluent |
| Lactose 200M | 501.25 | Diluent |
| Povidone K-30 | 68.75 | Binder |
| Sodium | 68.75 | Disintegrant |
| Croscarmellose | | |
| Magnesium Stearate | 27.50 | Glidant |
| Opadry II | 206.25 | Colorant |
| 85F205017 blue | | |
| Purified water* | 340 ml | Granulation liquid |

Analysis of the above formulations is shown below.

Active Tablets:

| Test | Specification |
|---|---|
| Hardness | ≥7.0 kp (≥69N) |
| Friability | ≤1.0% |
| Assay (HPLC) | Contain not less than 90.0% and |
| | not more than 110.0% of the |
| | labeled amount of Compound X |
| Total Degradation | ≤2.0% (a/a) |
| Product | |

### Comparative Example

The specifications for the placebo tablets are the same as those described above for Compound X tablets, with the exception that the identification of Compound X should be negative when the HPLC test for identification and assay is performed. Dissolution test is not performed and the “release” is controlled by means of a disintegration test. This is fully described in United States Pharmacopeia 33 Chapter <701> and European Pharmacopeia, 6^{th} Edition, 2.9.1.

The purity test was not performed for the placebo tablets.

| Test | Specification |
|---|---|
| Hardness | ≥7.0 kp (≥69N) |
| Friability | ≤1.0% |
| Disintegration | Not more than 30 minutes |
| Identification | Negative. |
| (HPLC) | No major peak in the chromatogram |
| | of the sample solution with |
| | retention time corresponding to |
| | that in the chromatogram of the |
| | standard solution. |
| | <5 mg/tablet |

From the presented development work the following conclusions can be made:
The wet granulation process gives rise to better granule and tablet physical properties than the direct compression process.
The colorant addition process which gives rise to better color homogeneity is the one process where the colorant is added in step (a), (b) or (c) of the wet granulation method.

In this experiment, the best combination of excipients is lactose and microcrystalline cellulose (Avicel 101), because they give rise to more evenly colored tablets without loss of good technological characteristics.

### Experimental Work Relating to Stress Studies

The following formulations were subjected to a stress study. In this stress study, the samples were prepared in duplicate. One sample was left at room temperature and the other was stored at 75° C./75% relative humidity for 18 days:

| | Composition | | | | |
|---|---|---|---|---|---|
| | IV | V | VI | VII | VIII |
| | Unit quantity | Unit quantity | Unit quantity | Unit quantity | Unit quantity |
| Material | (mg) | (mg) | (mg) | (mg) | (mg) |
| Compound X | 100.00 | 100.00 | 100.00 | 100.00 | 100.00 |
| (micronized) | | | | | |
| Microcrystalline | 54.75 | 54.75 | 54.75 | 54.75 | 54.75 |
| cellulose (MCC 101) | | | | | |
| Dibasic dihydrate | 54.40 | — | — | — | — |
| calcium phosphate | | | | | |
| (Emcompress ®) | | | | | |
| Isomalt (Isomalt 801) | — | 54.40 | — | — | — |
| Lactose 200M | — | — | 54.40 | — | — |
| Mannitol | — | — | — | 54.40 | — |
| Starch (Starch 1500) | — | — | — | — | 54.40 |
| Povidone K-30 | 11.75 | 11.75 | 11.75 | 11.75 | 11.75 |
| Sodium croscarmellose | 11.75 | 11.75 | 11.75 | 11.75 | 11.75 |
| Magnesium stearate | 2.35 | 2.35 | 2.35 | 2.35 | 2.35 |
| Purified water | 0.05 | 0.05 | 0.05 | 0.05 | 0.05 |
| Coating agent | 6% | 6% | 6% | 6% | 6% |
| | weight | weight | weight | weight | weight |
| | gain | gain | gain | gain | gain |
| Purified water coating | q ad for | q ad for | q ad for | q ad for | q ad for |
| (ml) | 20% | 20% | 20% | 20% | 20% |
| | solution | solution | solution | solution | solution |
| Total | 235.00 | 235.00 | 235.00 | 235.00 | 235.00 |

### Results:

| | Assay (% w/w) | | | Purity (Total degradation products) (% a/a) | | |
|---|---|---|---|---|---|---|
| Composition | 25° C. | 75° C. | Difference | 25° C. | 75° C. | Difference |
| IV | 99.3 | 90.5 | 8.8 | 0.2 | 0.1 | −0.1 |
| V | 101.2 | 95.3 | 5.9 | 0.4 | 0.1 | −0.3 |
| VII | 100.9 | 92 | 8.9 | 0.2 | 0.2 | 0 |
| VIII | 103.5 | 95.3 | 8.2 | 0.2 | 0.1 | −0.1 |

The best performance was observed in compositions V and VIII, which used as fillers Isomalt and starch 1500 respectively. Composition V, which used Isomalt as a filler presented a higher disintegration time (14 min 39 s). For this reason it was decided to proceed with a formulation based on composition VIII, but with a 200 mg strength and without coating, which was afterwards subjected to the following variations and studied in a new stress study:
Composition IX: composition equivalent to composition VIII but with a 200 mg strength instead of 100 mg
Composition XIII: variation of composition IX using a different superdisintegrant (crospovidone)
Composition X: variation of composition IX using a different lubrication system (Talc and Colloidal Hydrated Silica)

| | Composition | | | |
|---|---|---|---|---|
| | VIII | IX | XIII | X |
| | Unit quantity | Unit quantity | Unit quantity | Unit quantity |
| Material | (mg) | (mg) | (mg) | (mg) |
| Compound X | 100.00 | 200.00 | 200.00 | 200.00 |
| (micronized) | | | | |
| Microcrystalline | 54.75 | 109.50 | 109.50 | 109.50 |
| cellulose (MCC 101) | | | | |
| Talc | — | — | — | 5.36 |
| Colloidal | — | — | — | 5.36 |
| Hydrated Silica | | | | |
| Crospovidone | — | — | 23.50 | — |
| Starch 1500 | 54.40 | 108.80 | 108.80 | 108.80 |
| Povidone K-30 | 11.75 | 23.50 | 23.50 | 23.50 |
| Sodium croscarmellose | 11.75 | 23.50 | — | 23.50 |
| Magnesium stearate | 2.35 | 4.7 | 4.7 | — |
| Purified water | 0.05 | 0.05 | 0.05 | 0.05 |
| Coating agent | 6% weight | — | — | — |
| | gain | | | |
| Purified water coating | q ad for 20% | — | — | — |
| (ml) | solution | | | |
| Total | 235.00 | 470.00 | 470.00 | 476.02 |

### Results:

| | Assay (% w/w) | | | Purity (total degradation products) (% w/w) | | |
|---|---|---|---|---|---|---|
| | 25° C. | 75° C. | | 25° C. | 70° C. | |
| Composition | Assay | Assay | Difference | Purity | Purity | Difference |
| IX | 95.5 | 93.5 | 2.0 | 0.4 | 0.2 | −0.2 |
| XIII | 95.4 | 94.4 | 1.0 | 0.4 | 0.2 | −0.2 |
| X | 94.7 | 94.3 | 0.4 | 0.3 | 0.2 | −0.1 |

The results indicate that all the tested formulations are stable. All results were very positive as there was only a very small amount of active pharmaceutical ingredient (API) degradation.

Dissolution Studies

The following data was obtained from:
Formulations referenced herein as Compositions I, II, III and Batches 31, 36, 40-42:

### Test Conditions:

Paddle apparatus

### Volume: 1000 ml

### Medium: HCL 0.01M (pH 2.0)

Paddle speed: 100 rpm (5 mg); 75 rpm (25 and 100 mg)
Time: 45 minutes

### Dissolution Data:

5 mg—101% (100-103%)
25 mg—89% (88-92%)
100 mg—82% (76-94%)
Reference batch of API not formulated inside a capsule (Drug in capsule) (100 mg)—7% (6-8%)
Data indicate that all formulations presented a significant improvement in the dissolution profile over the API.

### Formulations Referenced Herein as Compositions IV to XVII:

### Test Conditions:

Paddle apparatus

### Volume: 1000 ml

Medium: acetate buffer pH 4.5+1% Sodium lauryl sulphate
Paddle speed: 75 rpm (50 mg); 100 rpm (200 mg)
Time: 45 minutes

### Dissolution Data:

50 mg—97% (94-99%)
200 mg—94% (93-95%)

### Formulations Referenced Herein as Compositions IV to XVII:

### Test Conditions:

Paddle apparatus

### Volume: 1000 ml

Medium: acetate buffer pH 4.5+0.5% Sodium lauryl sulphate
Paddle speed: 75 rpm
Time: 45 minutes

### Dissolution Data:

5 mg—95% (91-99%)
100 mg—49% (45-53%)
The 100 mg strength formulation was also tested under the following conditions:
Paddle apparatus

### Volume: 1000 ml

Medium: acetate buffer pH 4.5+1.0% Sodium lauryl sulphate
Paddle speed: 100 rpm
Time: 45 minutes

### Dissolution Data:

100 mg—70% (59-77%)

### Experimental Work Relating to Disintegration Studies

The disintegration time and other parameters were determined for a number of the previously detailed formulations:

### 5 mg Strength

Composition

| | I | XIV |
|---|---|---|
| | Quantity | Quantity |
| Component | (mg/unit) | (mg/unit) |
| Compound X | 5.00 | 5.00 |
| Opadry II 85F205017 blue | 38.50 | — |
| MCC 101 | 100.75 | 108.75 |
| Lactose 200M | 100.50 | — |
| Mannitol | — | 108.75 |
| Povidone K-30 | 13.75 | 12.50 |
| Croscarmellose sodium | 13.75 | — |
| Crospovidone | — | 12.50 |
| Magnesium stearate | 2.75 | 2.50 |
| Purified water* | qs | qs |
| TOTAL | 275 | 250 |
| Parameter | | |
| Average weight (mg) | 275 | 250 |
| Thickness (mm) | 4.6 | 4.5 |
| Hardness (kp) | 14 | 11 |
| Friability (%) | 0.1 | 0.1 |
| Disintegration time (s) | 769 | 689 |

*does not appear in the final product

### 25 mg Strength

Composition

| | II | XV |
|---|---|---|
| | Quantity | Quantity |
| Component | (mg/unit) | (mg/unit) |
| Compound X | 25.00 | 25.00 |
| Opadry II 85F205017 blue | 38.50 | — |
| MCC 101 | 90.75 | 98.75 |
| Lactose 200M | 90.50 | — |
| Mannitol | — | 98.75 |
| Povidone K-30 | 13.75 | 12.50 |
| Croscarmellose sodium | 13.75 | — |
| Crospovidone | — | 12.50 |
| Magnesium stearate | 2.75 | 2.50 |
| Purified water* | qs | qs |
| TOTAL | 275 | 250 |
| Parameter | | |
| Average weight (mg) | 275 | NA |
| Thickness (mm) | 4.6 | NA |
| Hardness (kp) | 12 | NA |
| Friability (%) | 0.2 | NA |
| Disintegration time (s) | 887 | NA |

*does not appear in the final product

### 50 mg Strength

| | Composition |
|---|---|
| | XI |
| | Quantity |
| Component | (mg/unit) |
| Compound X | 50.00 |
| MCC 101 | 184.70 |
| Pregelatinized starch | 183.60 |
| Povidone K-30 | 23.50 |
| Crospovidone | 23.50 |
| Magnesium stearate | 4.70 |
| Purified water* | Qs |
| TOTAL | 470 |
| Parameter | |
| Average weight (mg) | 470 |
| Thickness (mm) | 4.6 |
| Hardness (kp) | 6 |
| Friability (%) | 0.1 |
| Disintegration time (s) | 364 |

*does not appear in the final product

### 100 mg Strength

| | Composition | | |
|---|---|---|---|
| | III | XII | XVI |
| | Quantity | Quantity | Quantity |
| Component | (mg/unit) | (mg/unit) | (mg/unit) |
| Compound X | 100.00 | 100.00 | 100.00 |
| Opadry II 85F205017 blue | 38.50 | — | — |
| MCC 101 | 53.25 | 159.70 | 61.25 |
| Lactose 200M | 53.00 | — | — |
| Mannitol | — | — | 61.25 |
| Pregelatinized starch | | 158.6 | |
| Povidone K-30 | 13.75 | 23.50 | 12.50 |
| Croscarmellose sodium | 13.75 | — | — |
| Crospovidone | — | 23.50 | 12.50 |
| Magnesium stearate | 2.75 | 4.70 | 2.50 |
| Purified water* | qs | qs | qs |
| TOTAL | 275 | 470 | 250 |
| Parameter | | | |
| Average weight (mg) | 275 | NA | 250 |
| Thickness (mm) | 4.7 | NA | 4.6 |
| Hardness (kp) | 15 | NA | 15 |
| Friability (%) | 0.3 | NA | 0.1 |
| Disintegration time (s) | 693 | NA | NA |

*does not appear in the final product;

### 200 mg Strength

Composition

| | XIII | XVII |
|---|---|---|
| | Quantity | Quantity |
| Component | (mg/unit) | (mg/unit) |
| Compound X | 200.00 | 200.00 |
| MCC 101 | 109.50 | 11.25 |
| Pregelatinized starch | 108.80 | — |
| Mannitol | — | 11.25 |
| Povidone K-30 | 23.50 | 12.50 |
| Crospovidone | 23.50 | 12.50 |
| Magnesium stearate | 4.70 | 2.50 |
| Purified water* | Qs | qs |
| TOTAL | 470 | 250 |
| Parameter | | |
| Average weight (mg) | 470 | 250 |
| Thickness (mm) | 4.5 | 4.8 |
| Hardness (kp) | 9 | 13 |
| Friability (%) | 0.0 | 0.3 |
| Disintegration time (s) | 563 | 629 |

*does not appear in the final product

Additional formulations were prepared and the dissolution profile, bulk density. hardness and disintegration times were characterised. Suitable methods for determining bulk density are known in the art, for example, as detailed in the European Pharmacopeia, 6th Edition Test 2.9.15 “apparent volume”, pages 285-286, EDQM, 2007, or USP 31, vol. 1 test <616> page 231-232, The United States Pharmacopeia Convention, 2008.

Compositions XVIII to XX:

| | Composition | | | |
|---|---|---|---|---|
| | Placebo | XVIII | XIX | XX |
| | Unit quantity | Unit quantity | Unit quantity | Unit quantity |
| Material | (mg) | (mg) | (mg) | (mg) |
| Compound X | — | 25.00 | 100.00 | 200.00 |
| (micronized) | | | | |
| Microcrystalline | 160.00 | 142.00 | 86.00 | 11.90 |
| cellulose (MCC 101) | | | | |
| Isomalt 801 | 56.00 | 49.00 | 30.00 | 4.10 |
| Povidone K-30 | 12.50 | 12.50 | 12.50 | 12.50 |
| Sodium croscarmellose | 19.00 | 19.00 | 19.00 | 19.00 |
| Magnesium stearate | 2.50 | 2.50 | 2.50 | 2.50 |
| Total | 250.00 | 250.00 | 250.00 | 250.00 |

| | | | | |
|---|---|---|---|---|
| Formula | Placebo | XVIII | XIX | XX |

Final blend

| | | | | |
|---|---|---|---|---|
| Bulk density (g/ml) | 0.53 | 0.56 | 0.63 | 0.68 |

Tablets

| | | | | |
|---|---|---|---|---|
| Average weight (mg) | 248.0 | 248.8 | 250.2 | 249.7 |
| Hardness (kp) | 8.1 | 4.5 | 6.6 | 15.6 |
| Thickness (mm) | 4.09 | 4.06 | 4.23 | 4.48 |
| Friability (%) | 0.17 | 0.46 | 0.29 | 0.22 |
| Disintegration time (s) | 114 | 278 | 360 | 416 |
| Mass uniformity RSD (%) | 1.36 | 1.14 | 1.34 | 1.28 |

The following data was obtained from Compositions XVIII to XX:

### Test Conditions:

Paddle apparatus

### Volume: 1000 ml

Medium: acetate buffer pH 4.5+0.5% Sodium lauryl sulphate
Paddle speed: 100 rpm (25, 100 and 200 mg)
Time: up to 60 minutes

| | | | | API in capsule |
|---|---|---|---|---|
| | 25 mg | 100 mg | 200 mg | (100 mg DIC) |
| Time (min.) | Average % | Average % | Average % | Average % |
| 0 | 0 | 0 | 0 | 0 |
| 5 | 21 | 16 | 25 | 0.4 |
| 10 | 45 | 35 | 46 | 1.5 |
| 15 | 60 | 49 | 58 | 4.7 |
| 20 | 70 | 59 | 67 | 7.6 |
| 30 | 76 | 69 | 76 | 12 |
| 45 | 84 | 78 | 85 | 18 |
| 60 | 87 | 85 | 89 | 23 |

Data indicate that all formulations presented a significant improvement in the dissolution profile over the API.

Compositions XXI to XXIII:

| | Composition | | | |
|---|---|---|---|---|
| | Placebo | XXI | XXII | XXIII |
| | Unit quantity | Unit quantity | Unit quantity | Unit quantity |
| Material | (mg) | (mg) | (mg) | (mg) |
| Compound X | — | 25.00 | 100.00 | 200.00 |
| (micronized) | | | | |
| Microcrystalline | 132.00 | 116 | 70 | 9.5 |
| cellulose (MCC 101) | | | | |
| Dibasic dihydrate | 83.00 | 74 | 45.00 | 5.8 |
| calcium phosphate | | | | |
| Povidone K-30 | 12.50 | 12.50 | 12.50 | 12.50 |
| Sodium croscarmellose | 19.00 | 19.00 | 19.00 | 19.00 |
| Magnesium stearate | 3.50 | 3.50 | 3.50 | 3.5 |
| Total | 250.00 | 250.00 | 250.00 | 250.00 |

| | | | | |
|---|---|---|---|---|
| Formula | Placebo | XVIII | XIX | XX |

Final blend

| | | | | |
|---|---|---|---|---|
| Bulk density (g/ml) | 0.53 | 0.56 | 0.63 | 0.68 |

Tablets

| | | | | |
|---|---|---|---|---|
| Average weight (mg) | 254.4 | 251.1 | 250.0 | 251.5 |
| Hardness (kp) | 10.0 | 4.5 | 6.8 | 15.1 |
| Thickness (mm) | 4.12 | 4.10 | 4.22 | 4.49 |
| Friability (%) | 0.11 | 0.46 | 0.23 | 0.26 |
| Disintegration time (s) | 115 | 155 | 310 | 424 |
| Mass uniformity RSD (%) | 0.59 | 1.40 | 1.45 | 1.52 |

The following data was obtained from Compositions XXI to XXIII:

### Test Conditions:

Paddle apparatus

### Volume: 1000 ml

Medium: acetate buffer pH 4.5+0.5% Sodium lauryl sulphate
Paddle speed: 100 rpm (25, 100 and 200 mg)
Time: up to 60 minutes

| | | | | API in capsule |
|---|---|---|---|---|
| | 25 mg | 100 mg | 200 mg | (100 mg DIC) |
| Time (min) | Average % | Average % | Average % | Average % |
| 0 | 0 | 0 | 0 | 0 |
| 5 | 29 | 23 | 25 | 0.4 |
| 10 | 40 | 38 | 46 | 1.5 |
| 15 | 48 | 44 | 58 | 4.7 |
| 20 | 53 | 52 | 67 | 7.6 |
| 30 | 61 | 62 | 76 | 12 |
| 45 | 70 | 72 | 85 | 18 |
| 60 | 74 | 77 | 89 | 23 |

Data indicate that all formulations presented a significant improvement in the dissolution profile over the API.

It will be appreciated that the invention described above may be modified within the scope of the attached claims.

1. A pharmaceutical formulation comprising Compound X: 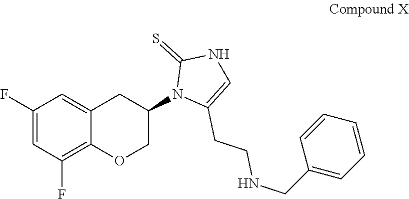 [SMILES: Fc1cc(F)c2c(c1)C[C@@H](n1c(CCNCc3ccccc3)c[nH]c1=S)CO2]
or a pharmaceutically acceptable salt or solvate thereof, in combination with one or more pharmaceutically acceptable excipients.

2. The pharmaceutical formulation according to claim 1, wherein the formulation exhibits a dissolution of at least about 50% at about 45 minutes, when measured at a temperature of about 37° C.±0.5° C. and a pH of about 4.5+0.5% sodium lauryl sulphate using a paddle apparatus.

3. The pharmaceutical formulation according to claim 1 having a bulk density of at least about 0.5 g/ml.

4-10. (canceled)

11. The pharmaceutical formulation according to claim 1, comprising (by weight of the total formulation excluding any coating present) from about 0.5 to about 90 wt % of Compound X or a pharmaceutically acceptable salt or solvate thereof, from about 4 to about 95 wt % of filler, from about 1 to about 20 wt % of binder, from about 2 to about 20 wt % of disintegrant and from about 0.5 to about 10 wt % of lubricant.

12. The pharmaceutical formulation according to claim 1, comprising (by weight of the total formulation excluding any coating present) from about 1 to about 85 wt % of Compound X or a pharmaceutically acceptable salt or solvate thereof, from about 7 to about 90 wt % of filler, from about 2 to about 15 wt % of binder, from about 2 to about 15 wt % of disintegrant and from about 0.5 to about 5 wt % of lubricant.

13. The pharmaceutical formulation according to claim 1, comprising (by weight of the total formulation excluding any coating present) from about 1 to about 85 wt % of Compound X or a pharmaceutically acceptable salt or solvate thereof, from about 7 to about 90 wt % of filler, from about 2 to about 10 wt % of binder, from about 2 to about 10 wt % of disintegrant and from about 0.5 to about 4 wt % of lubricant.

14. The pharmaceutical formulation according to claim 1, comprises a tiller selected from the group consisting of lactose, microcrystalline cellulose, dibasic dihydrate calcium phosphate, isomalt, mannitol or any, combination thereof, and/or a lubricant selected from the group consisting of magnesium stearate, talc, colloidal hydrated silica and any combination thereof, a disintegrant selected from the group consisting of crospovidone, sodium croscarmellose, sodium starch glycolate and any combination thereof, and/or a binder selected from the group consisting of povidone, pre-gelatinized starch, HPMC or any combination thereof.

15-17. (canceled)

18. The pharmaceutical formulation according to claim 1, comprising Compound X or a pharmaceutically acceptable salt or solvate thereof, anhydrous lactose, microcrystalline cellulose, sodium croscarmellose, povidone, magnesium stearate and optionally a colouring agent.

19. The pharmaceutical formulation according to claim 1, comprising Compound X or a pharmaceutically acceptable salt or solvate thereof, microcrystalline cellulose, crospovidone, pre-gelatinized starch, povidone and magnesium stearate.

20. The pharmaceutical formulation according to claim 1, comprising Compound X or a pharmaceutically acceptable salt or solvate thereof, microcrystalline cellulose, pre-gelatinized starch, sodium croscarmellose, povidone and magnesium stearate.

21. The pharmaceutical formulation according to claim 1, comprising Compound X or a pharmaceutically acceptable salt or solvate thereof, microcrystalline cellulose, pre-gelatinized starch, sodium croscarmellose, talc and colloidal hydrated silica.

22. The pharmaceutical formulation according to claim 1, comprising Compound X or a pharmaceutically acceptable salt or solvate thereof, microcrystalline cellulose, mannitol, crospovidone, povidone and magnesium stearate.

23. The pharmaceutical formulation according to claim 1, comprising Compound X or a pharmaceutically acceptable salt or solvate thereof, microcrystalline cellulose, isomalt, povidone, sodium croscarmellose and magnesium stearate.

24. The pharmaceutical formulation according to claim 1, comprising Compound X or a pharmaceutically acceptable salt or solvate thereof, microcrystalline cellulose, dibasic dihydrate calcium phosphate, povidone, sodium croscarmellose and magnesium stearate.

25. The pharmaceutical formulation according to claim 1, comprising from about 1 mg to about 400 mg of Compound X or a pharmaceutically acceptable salt or solvate thereof.

26. The pharmaceutical formulation according to claim 1 for oral administration.

27. The pharmaceutical formulation according to claim 1 in the form of a tablet or capsule.

28. A method of treating pulmonary arterial hypertension comprising administering to a patient in need thereof a therapeutically effective amount of the pharmaceutical formulation according to claim 1.

29. The pharmaceutical formulation according to claim 1 for use in the treatment of pulmonary arterial hypertension.

30. (canceled)

31. The method of preparing the pharmaceutical formulation according to comprising the steps of:
(a) mixing Compound X or a pharmaceutically acceptable salt or solvate thereof: 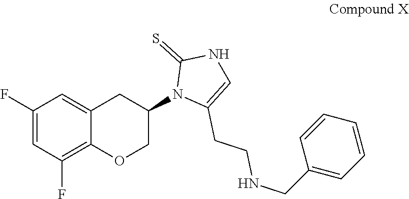 [SMILES: Fc1cc(F)c2c(c1)C[C@@H](n1c(CCNCc3ccccc3)c[nH]c1=S)CO2]
with at least one excipient;
(b) mixing the mixture from step (a) with at least one further excipient;
(c) adding at least one further excipient to the mixture from step (b) and mixing;
(d) adding at least one lubricant to the mixture from step (c) and mixing; and optionally
(e) pressing the mixture from step (d) to form a tablet, optionally with a predetermined weight.
